(19) United States
(12) Patent Application Publication
(10) Pub. No.: US 2023/0105463 A1
(43) Pub. Date: Apr. 6, 2023

(54) INTEGRATED DOSE COUNTER

(71) Applicant: Trudell Medical International, London (CA)

(72) Inventors: Adam Meyer, London (CA); Stephen Costella, London (CA); Neritan Alizoti, London (CA); Jake Desmarais, Toronto (CA); Atin Patel, Mississauga (CA); Jovin Basil, London (CA)

(73) Assignee: Trudell Medical International, London (CA)

(21) Appl. No.: 17/954,066

(22) Filed: Sep. 27, 2022

Related U.S. Application Data
(60) Provisional application No. 63/251,991, filed on Oct. 4, 2021.

(51) Int. Cl. A61M 15/00; G06M 1/10

(52) U.S. Cl. CPC A61M 15/0071; A61M 15/0025; G06M 1/105; A61M 2205/8206; A61M 2205/6063

(57) ABSTRACT

An indicating device includes a mechanical dose counter adapted to count the number of doses that have been dispensed from or remain in a container and an electronic module coupled to the mechanical dose counter and adapted to record when the doses have been dispensed from the container. Methods of using and assembling the device are also provided.

This application claims the benefit of U.S. Provisional Application No. 63/251,991, filed Oct. 4, 2021 and entitled “Integrated Dose Counter,” the entire disclosure of which is hereby incorporated herein by reference.

### TECHNICAL FIELD

The present invention relates generally to a dose counter, and in particular to an integrated dose counter including a mechanical dose counter and an electronic module, and also to methods of delivering aerosol medicament or the like and methods of assembling the integrated dose counter.

### BACKGROUND

Metered dose inhalers (MDI's) are not reusable devices and so are disposed of after their prefilled number of doses have been administered or have exceeded the specified shelf life of the drug. Mechanical dose counting mechanisms may be integrated with MDI's, and may be required by the FDA. There are two common types of mechanical dose counters that use either (1) displacement of the MDI canister relative to the actuator or (2) force applied to the MDI as the means of detecting and therefore counting an actuation or release of a dose. Mechanical dose counters typically are only able to provide information about the number of doses dispensed from or remaining in the container, and typically do not provide information about how the dose was taken or when.

### BRIEF SUMMARY

In one aspect, one embodiment of an indicating device includes a mechanical dose counter adapted to count the number of doses that have been dispensed from or remain in a container and an electronic module coupled to the mechanical dose counter and adapted to record when the doses have been dispensed from the container.

In another aspect, one embodiment of a method of assembling a medicament dispensing device includes coupling a mechanical dose counter adapted to count the number of doses that have been dispensed from or remain in a container to an electronic module adapted to record when the doses have been dispensed from the container, coupling at least one of the mechanical dose counter or the electronic module to the container or an actuator housing, and coupling the container to the actuator housing.

In another aspect, a method of counting a dose dispensed from a medicament dispensing device includes pushing one of a mechanical dose counter or an electronic module coupled to the mechanical dose counter, wherein at least one of the mechanical dose counter and electronic module are coupled to a container or an actuator housing holding the container, dispensing a dose of medicament from the container, counting the dose of medicament dispensed from the container with the mechanical dose counter and displaying the number of doses that have been dispensed from or remain in the container with the mechanical dose counter, and recording when the dose of medicament was dispensed from the container with the electronic module.

In another aspect, an indicating device includes a mechanical dose counter adapted to count the number of doses that have been dispensed from or remain in a container. A printed circuit board assembly may be adapted to record when the doses have been dispensed from the container. A battery may be electrically connectable to the printed circuit board assembly. A retaining member may be coupled to the mechanical dose counter, and sandwiches the printed circuit board assembly between the retaining member and the mechanical dose counter.

In yet another aspect, an indicating device includes a mechanical dose counter adapted to count the number of doses that have been dispensed from or remain in a container. The mechanical dose counter may include a base and a cap moveable relative to the base between a first position and a second position. A printed circuit board assembly may be adapted to record when the doses have been dispensed from the container. A battery may be connectable to the printed circuit board assembly. A contact mechanism may be disposed between the battery and the printed circuit board, wherein the contact mechanism is moveable between a first position wherein the battery is not electrically coupled to the printed circuit board assembly and a second position wherein the battery is electrically coupled to the printed circuit board.

### BRIEF DESCRIPTION OF THE DRAWINGS

FIG. 1 is a perspective and side view of a metered dose inhaler with a mechanical dose counter coupled to a container.

FIG. 2 are side views of a mechanical dose counter and electronic module applied to a metered dose inhaler.

FIG. 3 are views of the electronic module.

FIG. 4 is a side view of a metered dose inhaler with a mechanical dose counter and electronic module being actuated.

FIG. 5 is an exploded view of one embodiment of a force sensitive resistor assembly.

FIG. 6 is a side view of one embodiment of a mechanical dose counter and electronic module applied to a metered dose inhaler.

FIG. 7 is a side view of one embodiment of a mechanical dose counter and electronic module applied to a metered dose inhaler.

FIG. 8 is a side view of one embodiment of a mechanical dose counter and electronic module applied to a metered dose inhaler.

FIG. 9 is a side view of one embodiment of an electronic module applied to a metered dose inhaler.

FIG. 10 is a side view of one embodiment of an electronic module applied to a metered dose inhaler.

FIG. 11 is a cross-sectional view of one embodiment of a mechanical dose counter coupled to an electronic module.

FIG. 12 is a cross-sectional view of one embodiment of a mechanical dose counter coupled to an electronic module.

FIGS. 13 and 14 are a cross-sectional view of a metered dose inhaler with a mechanical dose indicator applied thereto.

FIGS. 15-18 are cross-sectional views of one embodiment of a mechanical dose counter being actuated.

FIG. 19 is a cross-sectional view of one embodiment of a mechanical dose counter.

FIG. 20 is a cross-sectional view of one embodiment of a mechanical dose counter.

FIG. 21 is a flow chart showing the operation of one embodiment of an electronic module.

FIG. 22 is a flow chart showing the operation of one embodiment of an electronic module.

FIG. 23 is a flow chart showing the operation of one embodiment of an electronic module.

FIG. 24 is a schematic diagram of one embodiment of a system.

FIG. 25 are views of one embodiment of an electronic module.

FIG. 26 is an exploded view of one embodiment of an electronic module.

FIG. 27 is a flow chart showing a TX event for BLE advertisement.

FIG. 28 is a force sensitive resistor sensor assembly.

FIG. 29 is a cross-sectional view of a mechanical dose counter being applied to the bottom of a container.

FIG. 30 is a schematic illustrating a computer structure.

FIG. 31 is a schematic illustration of a communication system.

FIG. 32 is an exploded view of a mechanical dose counter.

FIG. 33 is a cross-sectional view of one embodiment of a wake-up switch.

FIG. 34 is a partial cross sectional view of one embodiment of a mechanical dose counter and electronic module applied to a metered dose inhaler.

FIG. 35 is a side perspective view of one embodiment of a mechanical dose counter and electronic module.

FIG. 36 is a bottom view of the mechanical dose counter and electronic module shown in FIG. 35.

FIG. 37 is a partial cross sectional view of another embodiment of a mechanical dose counter and electronic module applied to a metered dose inhaler.

FIG. 38 is a side perspective view of another embodiment of a mechanical dose counter and electronic module.

FIG. 39 is a bottom perspective view the mechanical dose counter and electronic module shown in FIG. 38.

FIG. 40 is a partial cross sectional view of another embodiment of a mechanical dose counter and electronic module applied to a metered dose inhaler.

FIG. 41A is a cross sectional side view of another embodiment of a mechanical dose counter and electronic module applied to a medicament container.

FIG. 41B is a partial cross sectional side view of the mechanical dose counter and electronic module applied to the medicament container in a metered dose inhaler assembly.

FIGS. 42A and B are top perspective and partial cross sectional side views of one embodiment of pressurized metered dose inhaler.

FIG. 43 is a side schematic view showing the communication between a pressurized metered dose inhaler, valved holding chamber and local computing device.

FIG. 44 is a schematic view showing communication between various smart devices.

FIG. 45 is a schematic view showing communication between various smart devices.

FIG. 46 is a schematic showing the electronic module architecture.

FIG. 47 is a flow chart showing the operation of one embodiment of the system.

FIG. 48 is flow chart showing the Accelerometer and Wake/Shake Detection Logic.

FIG. 49 is a flow chart showing the Microcontroller Logic.

FIG. 50 is a flow chart showing the Bluetooth and Advertisement Packet Logic.

FIG. 51 is a flow chart showing the IR detection an ADC block logic.

FIG. 52 is a schematic showing the finite state machine logic for the system.

FIG. 53 is an exploded perspective view of another embodiment of a mechanical dose counter and electronic module.

FIG. 54 is a top view of the mechanical dose counter and electronic module shown in FIG. 53.

FIG. 55 is a cross-sectional view of the mechanical dose counter and electronic module taken along line 55-55 of FIG. 54.

FIG. 56 is a cross-sectional view of the mechanical dose counter and electronic module taken along line 56-56 of FIG. 54.

FIG. 57 is a top view of a printed circuit board assembly.

FIG. 58 is a side view of the printed circuit board assembly shown in FIG. 57.

FIG. 59 is a bottom view of the printed circuit board assembly shown in FIG. 57.

FIG. 60 is a perspective view of a retaining member.

FIG. 61 is a top view of the retaining member shown in FIG. 60.

FIG. 62 is a cross-sectional view of the retaining member taken along line 62-62 of FIG. 61.

FIG. 63 is a cross-sectional view of the retaining member taken along line 63-63 of FIG. 61.

FIG. 64 is a perspective view of one embodiment of a coin battery.

FIG. 65 is a flow chart showing the operation of one embodiment of the system.

FIG. 66 is a flow chart showing another embodiment of the Bluetooth logic.

FIG. 67 is a top view of the mechanical dose counter and electronic module with a QR code applied thereto.

FIG. 68 is a side view of the mechanical dose counter and electronic module with a QR code applied thereto.

FIG. 69 is a flow chart illustrating the QR code operation.

FIG. 70 is a flow chart illustrating an alternative QR code operation.

FIG. 71 is a flow chart illustrating an alternative QR code operation.

FIG. 72A is a partial enlarged view of one embodiment of a contact mechanism in a first position.

FIG. 72B is a partial enlarged view of the contact mechanism in a second position.

FIG. 73A is a perspective view of a retaining member and printed circuit board assembly with an alternative embodiment of a contact mechanism.

FIG. 73B is an enlarged view of the contact mechanism shown in FIG. 73A in a first position.

FIG. 73C is an enlarged view of the contact mechanism shown in FIG. 73A in a second position.

FIG. 74A is a cross-sectional view of one embodiment of a mechanical dose counter and electronic module with another embodiment of a contact mechanism in a first position.

FIG. 74B is a cross-sectional view of one embodiment of a mechanical dose counter and electronic module with another embodiment of a contact mechanism in a second position.

FIG. 75 is a cross-sectional view of one embodiment of a mechanical dose counter and electronic module with another embodiment of a contact mechanism FIG.

FIG. 76A is a perspective view of one embodiment of a mechanical dose counter and electronic module with another embodiment of a contact mechanism.

FIG. 76B is a cross-sectional view of the mechanical dose counter and electronic module shown in FIG. 76A.

FIG. 77 is a top view of another embodiment of a retaining member and battery with another embodiment of contact mechanism.

FIG. 78A a partial top view of the contact mechanism shown in FIG. 77 in a first position.

FIG. 78B a partial top view of the contact mechanism shown in FIG. 77 in a second position.

FIG. 79A is a flow chart showing one embodiment of a shake detection algorithm.

FIG. 79B is a flow chart showing another embodiment of a shake detection algorithm.

FIG. 79C is a flow chart showing another embodiment of a shake detection algorithm.

FIG. 79D is a flow chart showing another embodiment of a shake detection algorithm.

FIG. 79E is a flow chart showing another embodiment of a shake detection algorithm.

FIG. 79F is a flow chart showing another embodiment of a shake detection algorithm.

### DETAILED DESCRIPTION OF THE DRAWINGS AND THE PRESENTLY PREFERRED EMBODIMENTS

### Overall Embodiment Description:

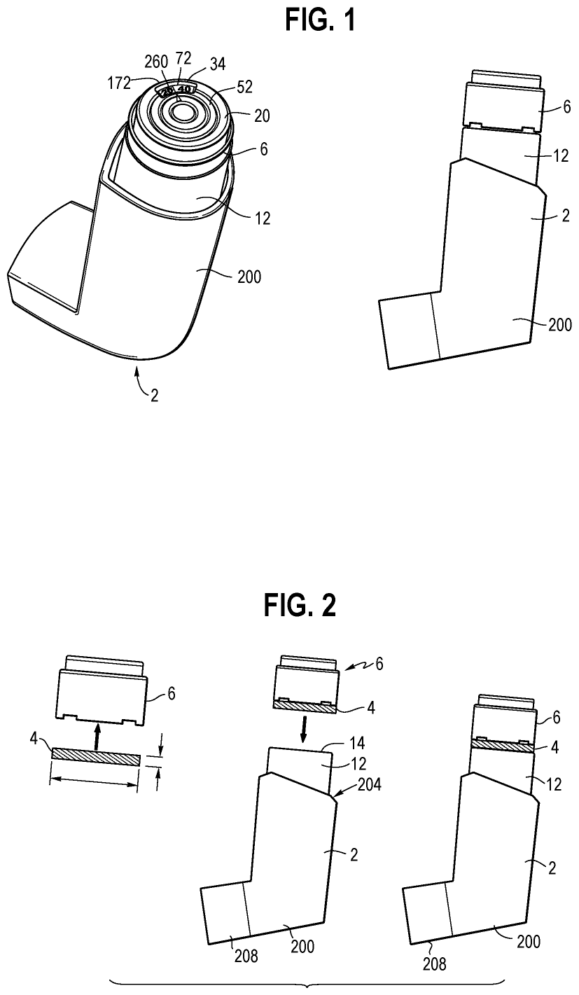

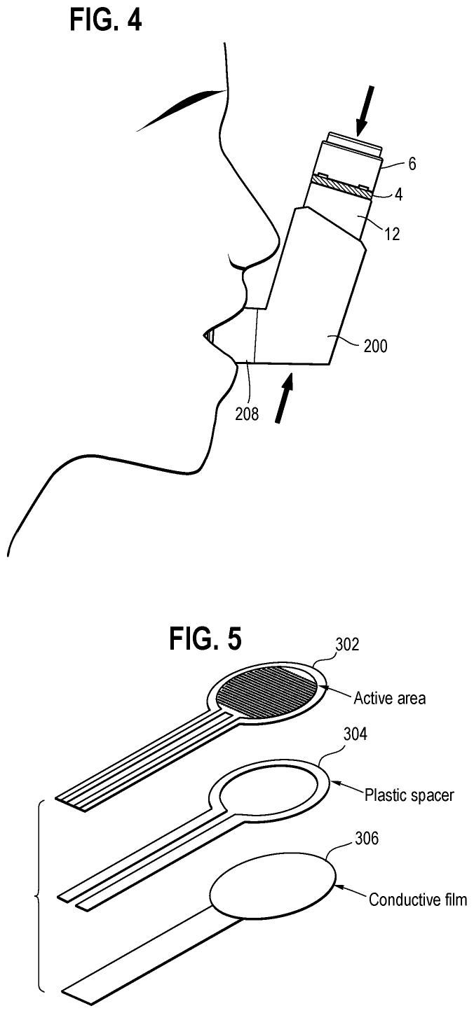

In one aspect, one embodiment of an indicating device provides mechanical dose counting and electronic dose counting, which would also enable additional information to be captured and communicated externally of a medication delivery device or system, including for example and without limitation a metered dose inhaler (MDI) 2, shown for example in FIGS. 1, 2 and 4. For example, such information may assist in determining whether a patient was adherent to their prescribed treatment where adherence can be described as persistence, whether the medication was taken and was taken at the right time, and whether there was compliance, i.e., was the medication taken properly.

The electronic dose counting and tracking module (EM) 4 is very low cost and may be easily integrated with existing MDI's that are already manufactured on a very low cost platform. Secondly, the electronics are developed with a very small form factor, which is an advantage in that would be easier to integrate into existing MDI's, provide greater flexibility in how they are integrated, and have the least impact on overall MDI functionality and usability. Together with low cost and size requirements, the selection of electronics and components have very low energy consumption requirements while accurately and reliably detecting, storing, and communicating each actuation of the dose counter.

In one embodiment, the EM 4 is permanently attached to an existing mechanical dose counter, for example a mechanical top mounted actuation indicator (TMAI) 6, forming the electronic TMAI (eTMAI) assembly. The resulting eTMAI may then be non-removeably coupled to the canister portion of a pressurized metered dose inhaler (pMDI) 2, for example by the pMDI manufacturer using an adhesive label wrap 600, 1600, 1602. The EM 4 provides additional connectivity, enhanced functionality, and adherence tracking to the existing TMAI 6, while maintaining the mechanical dose counting functionality.

### Preferred Embodiment—Electro-Mechanical Dose Counter and Tracker Description:

One mechanical dose counter is a TMAI 6, for example as manufactured by Trudell Medical International, which is a force type dose counter. Various examples of the dose counter are disclosed in U.S. Pat. Nos. 6,082,358, 6,926,002 and 8,074,594, the entire disclosures or which are hereby incorporated herein by reference. (No license, expressed or implied, is intended to be granted to either of these patents by reason of the incorporation by reference herein).

Referring to the drawings, and in particular FIGS. 1, 2, 4, 13-20, 34, 37, 40 and 42A and B, an aerosol dispenser is shown as including a housing 200, or actuator boot, and a container 12 disposed therein. The housing has a longitudinally extending cavity 202 shaped to receive the container. A top portion of the housing is generally open such that the container can be inserted in the housing through opening 204 and be installed therein with a bottom end 14 of the container protruding from the housing so as to be exposed to the user for actuation.

The terms “longitudinal” and “axial” as used herein are intended to indicate the direction of the reciprocal movement of the container relative to the housing, and of an indicating device cap member relative to a base member. The terms “top,” “bottom,” “upwardly” and “downwardly” are intended to indicate directions when viewing the inhalation devices as shown in the Figures, but with the understanding that the container is inverted such that the top surface thereof is located adjacent the bottom of the housing and vice versa. Moreover, it should be understood that a user can use the container and dispenser in any number of positions, including but not limited to the preferred upright position shown in FIGS. 1, 2, 4, 13 and 14.

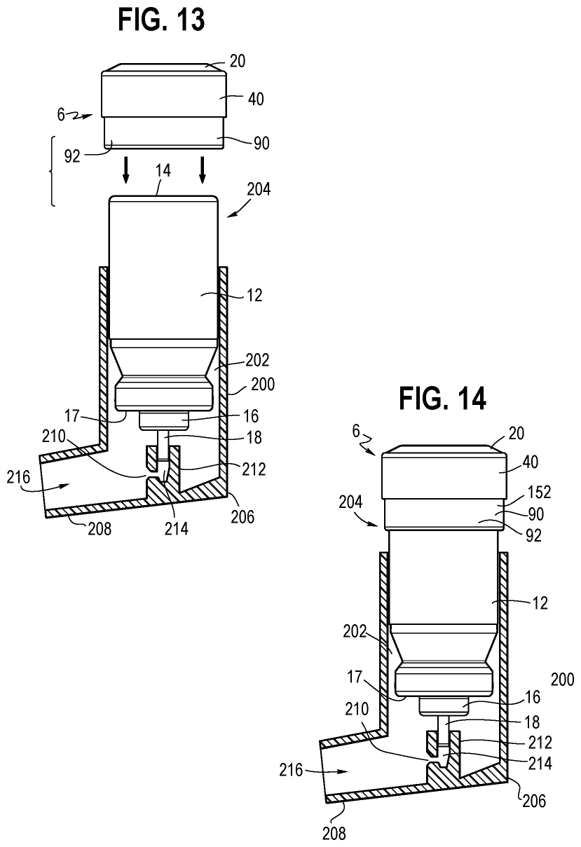

As shown in FIGS. 13 and 14, a cylindrical support block 212 having a well 214 is formed in a bottom portion 206 of the housing. An orifice 210 penetrates the support block to communicate with a bottom portion of the well. In one embodiment, a mouthpiece 208, intended for insertion into the mouth of a patient, forms an exhaust port 216 that communicates with the orifice and well. The mouthpiece 208 extends laterally from the housing so as to facilitate insertion of the mouthpiece into the mouth of the patient.

The container 12 is cylindrical and has a hub 16 disposed on a top surface 17 thereof. A valve stem 18 extends longitudinally from the hub. The valve stem extends coaxially from the container and is biased outwardly therefrom by a spring (not shown) mounted within the valve stem of the container. The container 12 is mounted in the housing by press fitting the valve stem 18 in the well 214 of the support block.

In a preferred embodiment, the container 12 is filled with a pressurized aerosol and medicament which is dispensed therefrom in specific metered doses by depressing or moving the valve stem 18 from an extended closed position to a depressed open position. A single metered dose is dispensed from the container by each reciprocal, longitudinal movement of the valve stem.

In operation, the opening of the valve stem is effected by moving the container 12 reciprocally within the housing 200 along a longitudinal axis, defined by the valve stem and the reciprocal movement of the container, by depressing the bottom end 14 of the container relative to the housing so as to move the valve stem 18 to the open position as it is supported within the well by the support block. As the valve stem is moved to the open position, the container dispenses a metered dose of aerosol and medicament through the well 214 and orifice 210. The aerosol and medicament are then transmitted to the patient through the exhaust port of the mouthpiece by way of either a self-generated or assisted airflow.

In other delivery systems, the housing and holder for the container are attached to a component having a chamber with an output end. Examples of these kinds of delivery systems are shown for example in U.S. Pat. No. 5,012,803, issued May 7, 1991, and U.S. Pat. No. 4,460,412, issued Sep. 11, 1984, both of which are hereby incorporated herein by reference. (No license, expressed or implied, is intended to be granted to either of these patents by reason of the incorporation by reference herein). In these kinds of delivery systems, the component having the chamber can be adapted to receive the mouthpiece of the housing, or it can be integrally connected with a holder supporting the container. In either embodiment, the metered dose of medicament in aerosol is first dispensed from the container into the chamber, and thereafter inhaled by the patient.

In a preferred embodiment, the container 12 is intended to dispense a predetermined number of metered doses of medicament. For example, conventional inhaler containers typically hold on the order of 100 to 200 metered doses. It should be understood, however, that the range of available doses could potentially vary from as few as one dose to as many as 500, or even more, depending, for example, on the capacity of the container, and/or the size of the metering dose valve. In operation, it can be important for the patient to be aware of the number of metered doses remaining in the container such that the patient is not caught unaware with an empty container when in need of the medicament.

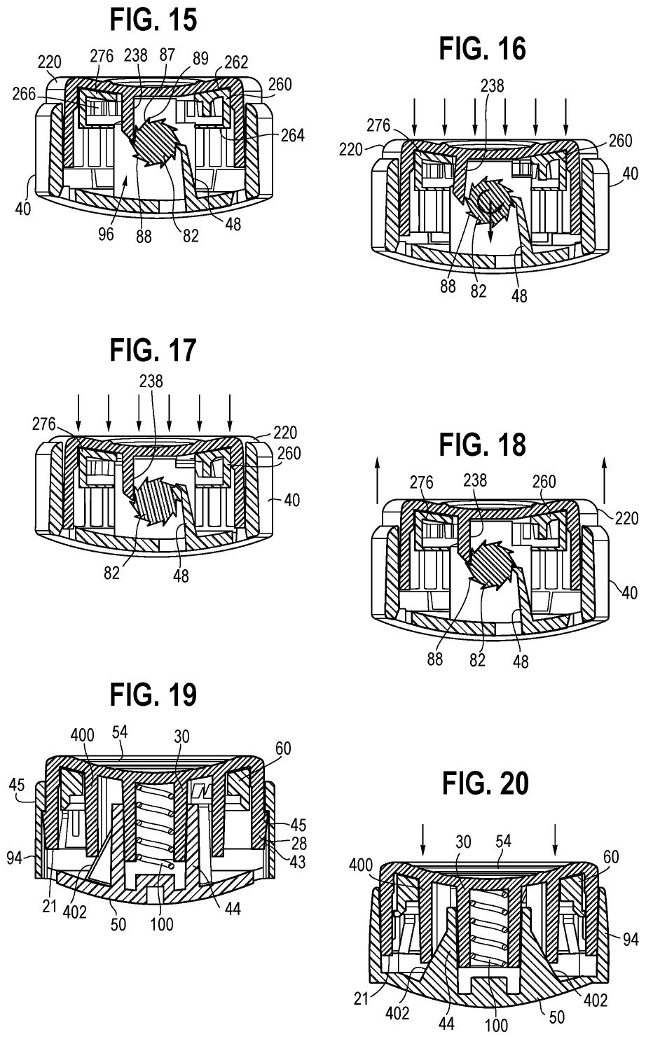

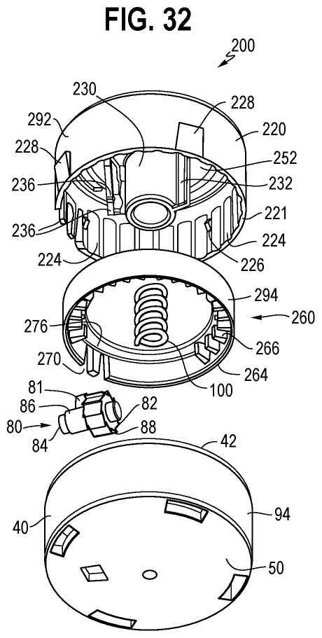

Now generally referring to the Figures, a mechanical dose indicating device 6 is shown. The indicating device 6 indicates the number of metered doses that have been dispensed from or remain in the container. As shown in the embodiments of FIGS. 1, 2, 4, 13-20 and 53-56, respectively, the indicating device 6 includes a cap member 20, 220, disposed in a base member 40. The base member 40 is configured such that it can be mounted to the bottom of the container 12. In a first embodiment, shown in FIGS. 15-18, 29 and 32, the base member includes a convex, or curved bottom portion 50, or floor, which is shaped to be received in and to mate with the bottom end 14 of the container, which has a concave or inwardly curved contour (see FIG. 29). The base member 40 is preferably bonded to the bottom of the container with adhesive, double sided tape, or similar bonding agent. Alternatively, a label 600, 1600, or other wrap component, may be wrapped around the base member and container, which have the same circumference in one embodiment. As shown in the embodiment of FIGS. 15-20 and 32, a circumferential skirt member 94 extends upwardly from the base portion to form a cavity 96.

Alternatively, as shown in FIGS. 13 and 14, the base member 90 includes a bottom portion, a downwardly depending circumferential skirt 152 and an upwardly depending circumferential skirt. Depending skirt 152 forms a recess or cavity which is shaped to receive the bottom end of the container 12. The base member 90 is mounted on the container either by bonding one or more of the bottom portion or skirt to the container, or by press fitting the container in the cavity so as to provide an interference fit between the container and the depending skirt. The upwardly depending skirt and bottom portion form an upper cavity overlying the lower cavity.

Although the disclosed container and indicating device, and in particular, the cap member and base member, are shown as preferably having a circular cross section, those skilled in the art should understand that the container and indicating device, including any adapter, can be configured in other shapes, including for example, but not limited to, a rectangular or triangular cross-section.

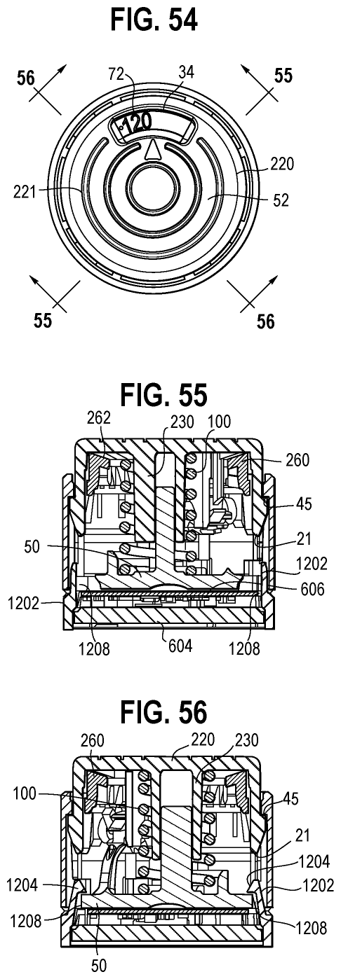

As best shown in FIGS. 1, 53 and 54, the cap member 20, 220 has a top portion 52 with a viewing window 34 formed therein. Preferably, the cap member 20 is circular and the viewing window is formed in the top portion adjacent the outer periphery of the cap member so as to overlie indicia applied to the top of an indicator member supported beneath the cap member. The viewing window can be configured in a number of various shapes. For example, the viewing window can be tapered, or it can be an arcuate shaped window bounded by coaxial inner and outer curved borders and radial side borders as shown in FIGS. 1 and 54. The top of the cap member preferably has a plurality of raised portions 221 or recesses forming a grippable pattern for the user's thumb, or finger. In this way, the user can firmly press down on the cap member without slippage. One of skill in the art should recognize that other patterns or grippable surfaces, such as a knurled pattern, can be applied to the cap member to facilitate the use of the indicating device.

Referring to FIGS. 13-20, 32 and 53, the cap member 20, 220 comprises a circumferential skirt 92, 292 depending downwardly from the top portion 52, 252. The skirt preferably has a smaller diameter than the upwardly depending skirt of the base member, such that the cap member skirt nests within the upwardly extending skirt of the base member. Alternatively, the cap member can be configured with a skirt having a larger diameter than the skirt of the base member such that the base member skirt nests in the cap member skirt. The cap member 20, 220 is moveably mounted to the base member 40 by way of a snap fit.

In particular, as shown in FIG. 19, the cap member includes a plurality of engagement members extending from an outer circumferential surface of the skirt that are captured in pockets formed along the inner circumferential surface of the base member skirt to form a snap-lock fit. In particular, the upper surface of the engagement member engages an engagement surface 45 defining the top of the pocket. In this way, the cap member is moveable with respect to the base member along an axial, or longitudinal, path. Alternatively, the rim of the base member can be curved slightly inward such that the engagement members engage the inwardly curved rim portion so as to prevent the cap member from being separated from the base member.

The axial movement of the cap member 20, 220 between a first position and a second position relative to the base member 40 is bounded or constrained by the engagement of the engagement members with the top of the base member pockets (or the base member rim) at a fully extended position and by engagement of a bottom rim 21, 221 of the cap member skirt with the upper surface of the bottom portion at the bottom of the stroke as shown in FIGS. 15-18. One of skill in the art should understand that the engagement members can alternatively be formed on the base member skirt so as to engage pockets or openings, or a rim (or like protrusion), formed on the cap member skirt.

As shown in FIGS. 15-19, 32 and 56, a spring 100 is disposed between the cap member and the base member. The spring is preferably disposed in a downwardly extending hub portion 30, 230 of the cap member and an upwardly extending hub portion 44 of the base member, which are received one in the other. Alternatively, a spring is disposed between the cap member and base member and is of such a size that the coils are positioned adjacent the inner circumferential surface of the cap member skirt. The spring 100 functions as a return mechanism and biases the cap member 60, 260 upwardly in the base member such that the engagement members 28, 228 of the cap member engage the upper portion of the pockets of the base member. Although a compression spring is shown in the Figures, it should be understood that a belleville washer, cantilever, torsion, leaf and/or tension springs would also work to bias the cap member upwardly into engagement with the base member. The springs can be made of metal or plastic.

As shown in FIG. 20, the return mechanism acting between the cap member and base member includes a plurality of resilient arm members 400 extending downwardly from the cap member. As the cap member is moved toward the base member, one or more of the arm members engages a ramped biasing surface 402 formed along an outer portion of the hub portion 44. The ramped biasing surface biases one or more of the resilient arm members outwardly as the cap member moves toward the base member. The resilient arm member(s) act as cantilever springs to bias the cap member away from the base member when the cap member is released by the user. One of skill in the art should understand that the resilient arm members can also be formed on the base member so as to engage a ramped surface formed on the cap member. One of skill in the art should also understand that the spring and resilient arm members can be used together, as shown in FIG. 20, or separately.

Referring to FIGS. 1, 15-20 32, 55 and 56, an indicator member 260 is rotatably mounted in the cap member 20, 220 about an axis substantially parallel to the axial movement of the cap member relative to the base member. The indicator member is generally open in the middle and includes a top portion 276 having an upper surface 262 that rotatably slides along a bottom surface of the top portion of the cap member. Alternatively, the indicator member can be mounted on the outside of the cap member with a viewing window formed in the indicator member for viewing indicia applied to the top of the cap member.

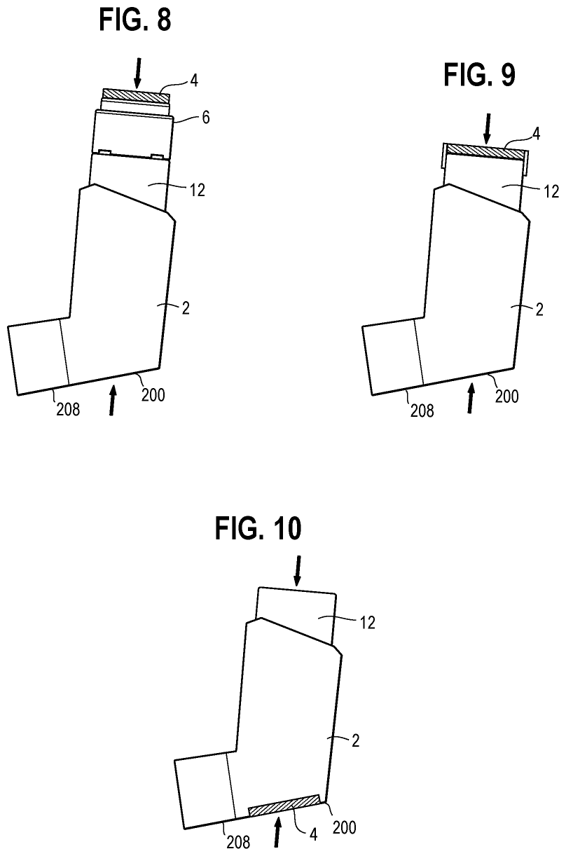

The indicator member 260 includes a circumferential skirt 274 depending downwardly from the top portion. Referring to FIGS. 5 and 8, a plurality of protrusions, or engagement tab members, extend from an inner circumferential surface of the cap member skirt and engage a rim 264 formed on the bottom of the indicator member skirt. Alternatively, the indicator member can include an engagement member, or rim, that engages a groove or similar opening in the cap member. In this way, the indicator member is secured to the cap member so as to prevent axial movement therebetween but where the indicator member is permitted to rotate relative to the cap member. The indicator member is installed by snap-fitting the indicator member within the cap member. One of skill in the art should understand that the indicator member could alternatively be rotatably mounted on the cap member hub portion (having a portion of the key member cut away), or on a similar axle secured to the cap member.

The indicator member 260 has a plurality of inwardly facing teeth 266 formed around the inner circumference of the skirt. The teeth are preferably formed about only a portion of the circumference.

The indicator member 60 includes a plurality of indentations 68 formed about the outer circumferential surface of the skirt 74. The cap member includes a pair of upwardly extending resilient indexing members 22, each having an end portion that engages one of the indentations so as to releasably engage the indicator member and prevent rotation therebetween. The angular distance between the indentations 68 is substantially the same as the angular distance between the plurality of indicator member teeth 66. In this way, the indexing member selectively engages the next indentation upon each incremental advancement of the indicator member defined by the distance between adjacent teeth.

Alternatively, the indentations and indexing member may be reversed, i.e., the indentations are formed about an inner circumferential surface of the cap member skirt and an indexing member depends downwardly from the indicator member in a void formed in the skirt of the indicator member.

As shown in FIGS. 1 and 54, dosage indicia 72 in the form of numbers or color codings are provided on the top surface of the indicator member and are visible to the user through the viewing window 34 provided in the top of the cap member. One of the skill in the art should understand that other indicia indicating the number of doses remaining in or dispensed from the container would include, but not be limited to, various alpha-numerical characters, words, terms or phrases (such as “full” and “empty”), scales, grids, arrows, raised portions, indentations, color coding and segmentation, shading and like markings, or any combination thereof. For example, a segmented color grid 172 displayed in the viewing window (as shown, e.g., in FIG. 1) could turn from green, indicating a full container, to yellow, indicating an intermediate capacity, and finally to red, indicating an empty container. It should also be understood that the indicia can be formed integrally with the counter member, or applied thereto by means of paint, dye, etching, pad printing, hot stamping or adhesive labels. When using numerical indicia, the numbers can be arranged to go from 0 (or some beginning number) to the predetermined number of available doses such that a display of that number to the user indicates that the container is empty, or, conversely, to go from the starting predetermined number to 0 (or some ending number), which again indicates to the user that the container is empty.

In a preferred embodiment, the indicator member is made of acrylonitrile butadiene styrene (“ABS”), which is receptive to certain alternative processes of printing or applying the indicia, including pad printing and hot stamping. The cap member and base member are preferably made of a hard plastic material such as Acetel.

Referring to FIGS. 15-20 and 32, a drive mechanism is shown as including a drive assembly 80 disposed between the cap and base. The drive assembly includes a ratchet wheel 82 coaxially mounted to a drive member on an axle 84. The ratchet wheel, drive member and axle can be made separately, with the ratchet wheel and drive member then mounted on the axle, or all three parts can be integrally molded as a one-piece component. The drive assembly is preferably made of hard plastic material such as Acetel.

The ratchet wheel 82 includes a plurality of teeth 88 (preferably ten) formed around its periphery. Each of the teeth includes an engagement surface 89 and a tapered surface 87. The drive member 86 includes a single tooth 81 extending radially from the axle 84. The drive assembly is mounted to the cap member by engaging opposite ends of the axle 84 with downwardly extending hub portions 36, 236 such that the axle, ratchet wheel and drive member rotate about an axis substantially perpendicular to the axial movement of the cap member relative to the base member and to the axis of rotation of the indicator member. Alternatively, the drive assembly can be mounted to the base member in a similar manner.

The drive mechanism further includes a pawl member 48, shown as a flexible rod or finger, which extends upwardly from the bottom portion of the base member and selectively engages one of the teeth of the ratchet wheel. Alternatively, the pawl member can be moveably secured to the cap member and extend through the base member to engage the top of the container, such that the axial movement of the cap member toward the container causes the pawl to move toward the ratchet wheel and engage one of the teeth thereon as described below. A non-return member 238, also shown as a flexible rod or finger, extends downwardly from the top portion of the cap member and selectively engages another of the teeth 88 of the ratchet wheel. It should be understood that the pawl member could alternatively extend from the cap member (and the non-return member from the base member) when the drive assembly is mounted to the base member, as described above.

In operation, as shown in FIGS. 15-18 and 32, the user depresses the cap 220 member from a fully extended (first) position (see FIG. 15) toward the base member such that the cap member bottoms out in the base member at the bottom of the stroke (FIG. 16) (second position) and such that the base member imparts an axial load on the container until a metered dosage is dispensed therefrom. In a preferred embodiment, the biasing force of the spring 100, or alternative return mechanism such as the resilient arm members which act as springs, is less than the biasing force of the spring located in the metering valve of the container, such that the cap member first bottoms out in the base member with the container then being moved downwardly in the housing until a metered dose is dispensed.

Referring to FIGS. 15-17, as the cap member 220 is depressed toward the base member 40, the pawl 48 selectively engages the engagement surface 89 of one of the ratchet wheel teeth and rotates the ratchet wheel. The tapered surface 87 of one of the teeth formed on the ratchet wheel simultaneously biases the non-return member 238 outwardly until it selectively engages the next tooth near the bottom of the stroke. The user then releases the cap member whereinafter the spring 100, or similar return mechanism, biases the cap member 220 away from the base member 40 until the engagement member engages the base portion at the top of the stroke as shown in FIG. 18. When the cap member is released by the user, the container is biased upwardly within the housing along the longitudinal axis such that the valve stem is moved to the closed position within the container. Simultaneously, as the cap member is released and allowed to move away from the base member, the pawl 48 is biased outwardly by the tapered surface 87 of one of the teeth on the ratchet wheel as the non-return member 238 prevents a backwards rotation thereof so as to maintain a unidirectional rotation of the ratchet wheel. At the top of the stroke (shown in FIG. 18), the pawl 48 is again placed in position for selective engagement with one of the teeth of the ratchet wheel. In this way, the ratchet wheel 82, and connected drive member 86, are advanced an incremental amount for every actuation of the container and the attendant release of medicament. The incremental amount is defined by and dependent on the number of teeth formed about the periphery of the ratchet wheel. When formed with ten teeth, as shown in the preferred embodiment, the ratchet wheel will make one full revolution for every ten actuations of the indicator device and container, or a tenth of a revolution for each actuation. One skilled in the art will appreciate that the ratchet wheel can be provided with various numbers of teeth formed about its periphery such that the more or less axial movements or actuations of the container are required to make one full rotation of the ratchet wheel. As can be appreciated the various movements of the ratchet and indexing portions of the drive and non-return mechanisms make various clicking noises during each actuation of the dose counter.

As noted, the mechanical dose counter or TMAI 6, and in particular the base 40, is attached to the top of an MDI, or bottom 14 of the container 12, in one embodiment and forms one half of the user interface of the MDI when integrated. The TMAI 6 is affixed to the MDI canister via a polymer label that is wrapped around both devices as shown in FIG. 1.

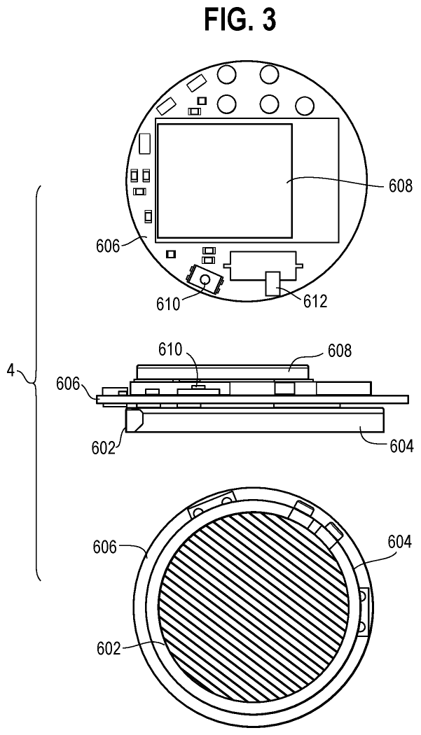

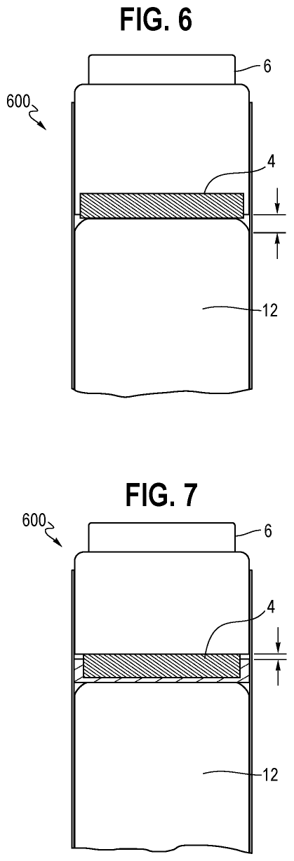

In one embodiment, an integrated dose indicator includes an electronic module 4 that may be combined with the mechanical dose counter, e.g., the TMAI 6, and may be mounted to the bottom of the TMAI via a fastening system such that the TMAI and electronics module become an assembly. Various fastener systems, or attachment devices, may include a modification of the bottom of the TMAI defining an extension, such as a circumferential skirt, that would house and contain the EM. Other attachment methods may include adhesives that to permanently or releasably attach the EM to the TMAI. The final eTMAI assembly would then be attached to the MDI for example with a polymer label wrap 600, 1600, 1602 as shown for example in FIGS. 6, 7, 41A and B, and 42A and B. To ensure that the EM is suitable for label wrapping, the existing method use to connect the TMAI to the MDI, the integrated TMAI and EM should have a maximum diameter no larger than the TMAI alone. Additionally, it may be desirable for the EM to be as short as possible (in the longitudinal direction) thus adding as little height as possible to the final integrated assembly. By minimizing the height increase of the overall assembly, and the distance between the actuation surfaces of the top of the TMAI and the bottom of the housing 200, the addition of the EM does not adversely affect the usability of the existing MDI. As mentioned above, the EM preferably remains within a maximum diameter defined by the diameter of the existing MDI canister and TMAI. Thus the height, which is preferably minimized, combined with the diameter maximum, defines the available volume and cylindrical shape to that volume within which the EM must be configured, as shown for example in FIG. 2.

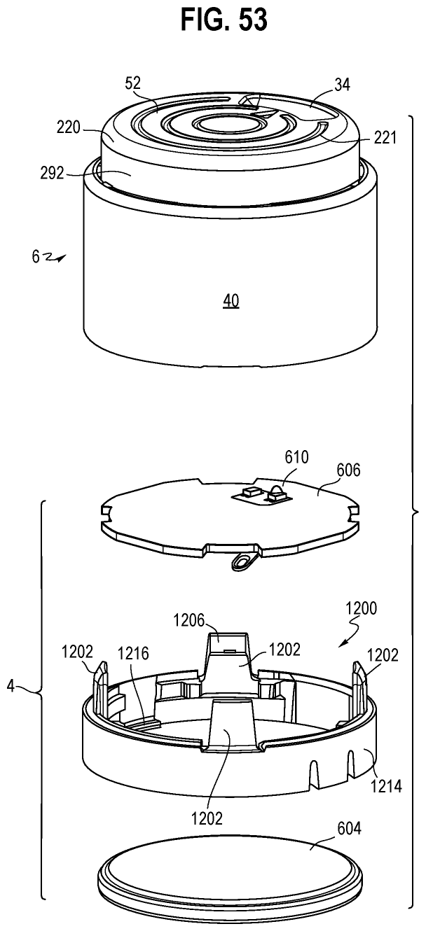

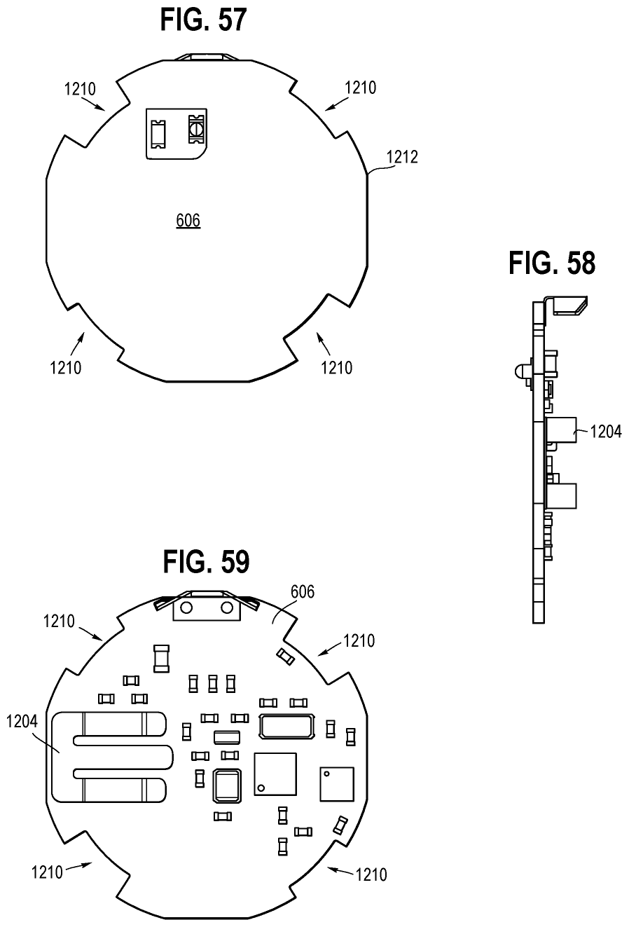

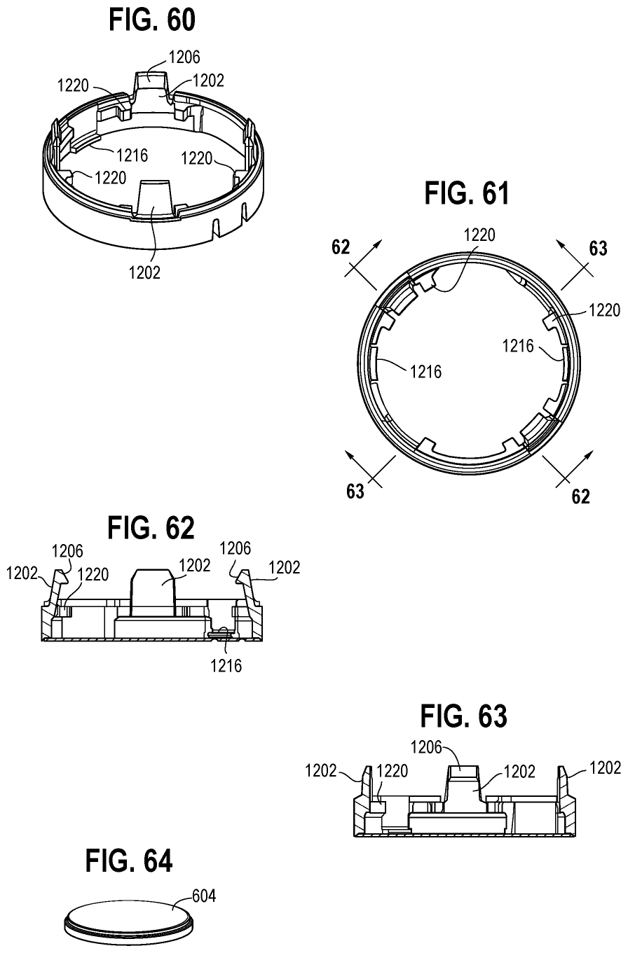

As shown in the embodiment of FIGS. 53-64, a retaining member 1200 secures the EM to the TMAI. In one embodiment, the retaining member 1200 is configured with a ring 1214 having a plurality of upstanding tabs 1202 extending upwardly from the ring. The ring 1214 may be configured in different sizes, for example with different diameters, to accommodate different size dose counters, for example with an outer surface that matches and lies flush with the exterior surface of the mechanical dose counter. At least one, and preferably a plurality, of the tabs 1202 are configured with a catch member 1206 at the end of the tab. For example, as shown in FIGS. 53 and 60, the retaining member 1200 includes four tabs 1202, with two of the tabs having a catch member 1206. The tabs 1202 are inserted through openings 1208 in the bottom or floor 50 of the base member 40. The tabs 1202 with catch members 1206 engage an edge of the bottom or floor 50 at the openings 1208 to secure the retaining member 1200 to the base member 40. One or more shelfs or supports 1216 extend radially inwardly from the ring 1214 and define a support surface facing the TMAI. A battery 604, shown as a coin battery, is inserted into the cavity defined by the ring and is supported by the shelf(s) or supports 1216. Above the battery, a printed circuit board assembly (PCBA) 606 is supported by the retaining ring, and in particular a plurality of supports 1220 that extend radially inwardly from the ring 1214. The PCBA 606 includes a plurality of cutouts 1210 extending radially inwardly from an outer peripheral edge 1212, with the cutouts 1210 being aligned with the tabs 1202 of the retaining member. During assembly, the retaining member 1200 is coupled to the base member 50 with the tabs 1202, with the PCBA 606 sandwiched between the retaining member, and held by the supports 1220, and the base member 50. The tabs 1202 extend through the cutouts 1210 and orient and prevent rotation of the PCBA. In other embodiments, the retaining member 1200 and PCBA 606 may be coupled with adhesive or other fastening mechanisms.

Referring to FIGS. 3, 5, 28, and 53-64, the electronic module may include one or more of the following elements:
1. Force sensor 602 for detecting the force applied to the system via the user's applied force to the top of the TMAI
2. Li-Ion Coin cell battery 604
3. Printed circuit board assembly (PCBA) 606
4. Bluetooth Low Energy module/transceiver 608 (e.g., Nordic chip)
5. LED 610
6. On/off switch 612
7. Infrared LED and Phototransistor (IR LED & PT)
8. Microcontroller (MCU) (within BTLE SoC)
9. Accelerometer
10. Battery contact 1204

### Electronic Module (EM) Description:

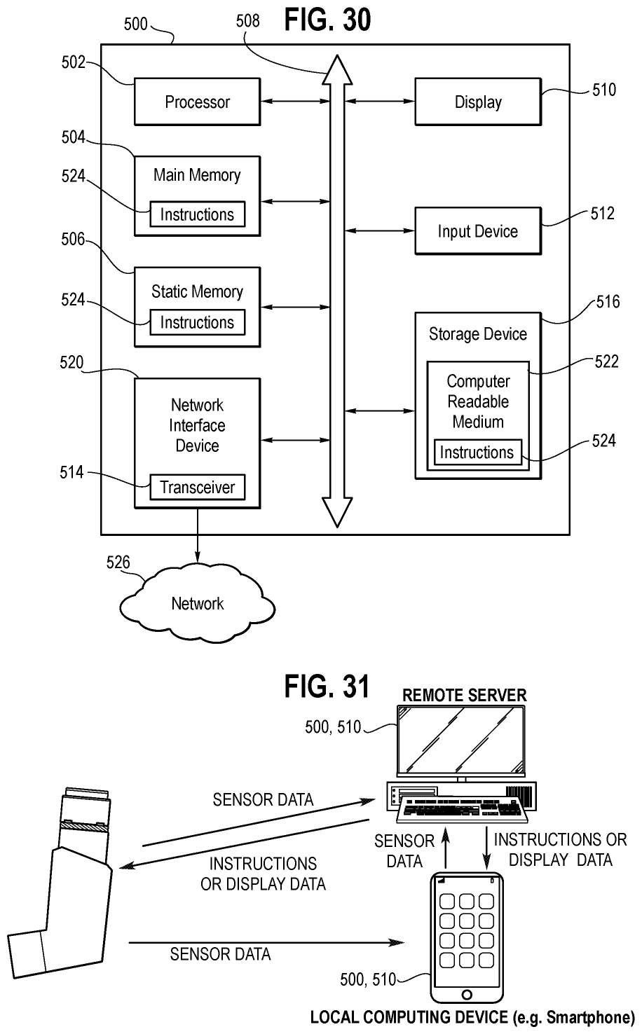

The EM registers an actuation when the MDI (and mechanical dose counter) are actuated (date and time) and stores this information. A wireless radio may be incorporated into the EM so that the actuation data stored in the device can be communicated to another device, preferably a smart phone where the data can be analyzed, processed, and presented to the patient or health care provider in a meaningful way via a software application (app), as shown for example in FIGS. 30 and 31. The EM may be affixed to the bottom of the TMAI, which may necessitate slight modifications to the TMAI but otherwise would leave the TMAI counter mechanism unchanged. In this way, the EM may be incorporated into existing MDI and TMAI systems, or provide for retrofitting such systems, such that proven TMAI mechanical dose counters do not need to be modified in any significant way in order to add the EM functionality. As well, manufacturing may be simplified by adding the EM module as a subassembly step without extensive alteration of the existing TMAI assembly process.

During operation, the user applies the actuation force to the top of the TMAI and the bottom of the MDI, which forces displacement in both the TMAI and MDI mechanisms. This is required for both devices to operate, i.e., for the TMAI to count and the MDI to release a dose of medication, as shown in FIG. 3.

The EM would receive the applied forces directed to it via the bottom of the TMAI and the top of the MDI, and the container in particular. In one embodiment, the force sensor may be configured as force sensitive resister (FSR) type sensor 602 which changes electrical resistance in response to pressure applied to the sensor. There are various suitable types of FSR sensors, with one preferred embodiment using a pressure sensitive conductive sheet (Velostat), shown in FIG. 28. FSR's are one embodiment of a preferred sensor configuration due to their low power consumption requirements. Due to the limitations on packaging (size) and cost, low power consumption is one consideration. Configured correctly within the design, a Velostat based FSR would be very low cost which would be advantageous over other more costly FSR type sensors. In this application, the Velostat material would be incorporated into the design between two conductive layers to form a sandwich. This sandwich would in effect form to the FSR as shown in FIG. 28. Alternate FSR's can be used including one configured as shown in FIG. 5, including an active area 302, a plastic spacer 304 and a conductive film 306. It should be understood that other force sensors may be used and are suitable for integration into the EM, including without limitation resistive, capacitive, piezo, load cell and/or Micro-Electro-Mechanical System MEMS force sensors, and/or combinations thereof. Referring to FIG. 45, in one embodiment, the eTMAI includes an EM and a TMAI architecture with various inputs and outputs, including BOL (beginning of operating life), FTA (Force to Actuate a TMAI, and FTF (Force to Fire a pMDI) inputs.

### Battery:

In one embodiment, the battery 604 in the EM may be a standard Coin Cell Lithium Ion (Li-ion) battery. Coin cell Li-ion batteries are readily available in volume quantities in configurations that are suitable to this application, and therefore provide lower cost battery options available for portable electronics. Secondly, coin cell Li-ion batteries have the energy storage capacity that is well suited to this application. Thirdly, the coin cell batter has a disc shape that is suitable for integration requirements. For example, the coin cell batteries may be configured in cylindrical formats in diameters matched to the TMAI and EM. In this way, a coin cell Li-ion battery may be selected that is as large as possible within the constraints of the maximum diameter defined by the TMAI/MDI so as to get maximum storage for the lowest possible height. The outside diameter of the TMAI/MDI Canister is normally between 22 mm and 24 mm. One specific battery that suits this application is the CR2012 (20 mm diameter, 1.2 mm height, and 50 mAh capacity). At 20 mm in diameter, the battery fits within the 22 mm diameter constraint of the TMAI/MDI canister leaving enough room for plastic walls to contain the assembly in the final configuration. This allows for label wrapping of the overall TMAI-EM assembly; the method currently used by manufacturers to couple the existing TMAI to the MDI canister. As shown in FIGS. 53-64, in one embodiment, the battery 604 is held against the battery contact 1204 of the PCBA 606 by the retaining member 1200.

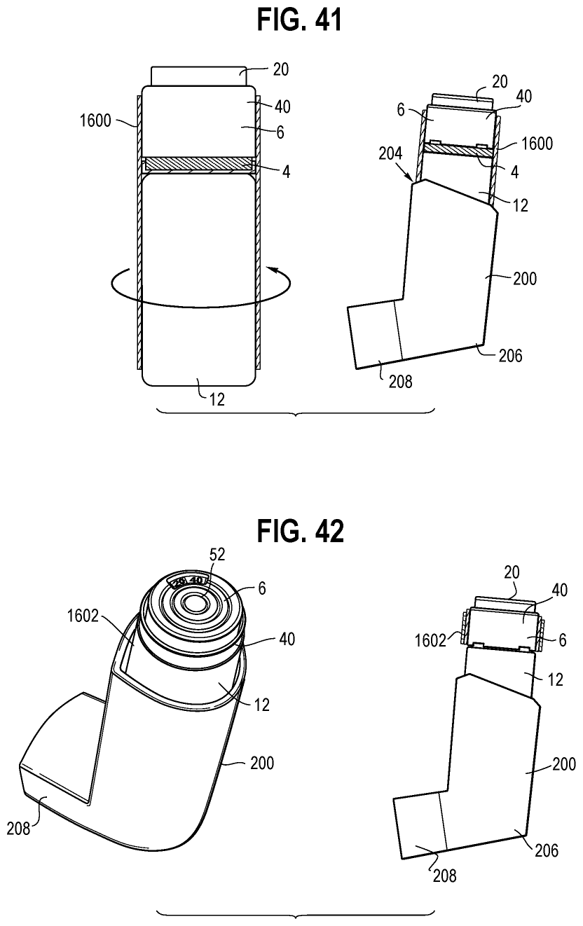

In an alternative embodiment, shown in FIGS. 41A and B and 42A and B, the battery 1600, 1602 is configured using imprint flexible battery technology that makes ultrathin, flexible, printed batteries. The battery 1600, 1602 may be configured as an imprint flexible 360° wrap around battery. The battery may be wrapped around the canister 12 like the label 1600 that attaches the eTMAI, 4, 6 to the container, or the battery wrap 1602 may be wrapped around the eMTAI components 4, 6 only and then covered by a label 600 connecting the dose counter to the container.

### Bluetooth Low Energy Transceiver:

Cost and size are similarly important in selecting an embodiment of the wireless transceiver 608 to be able to communicate with a Smartphone or tablet. As with selection of the sensor to detect actuation, low power consumption is a consideration in selecting the transceiver, together with the overall size or footprint to ensure that overall size of the package is minimized. One suitable embodiment uses a nRF24L01P 2.4 GHz Bluetooth Low Energy (BLE) transceiver 700 selected for low power consumption and minimal packaging dimensions for integration into the overall PCBA. While a BLE transceiver with only transmission capabilities is suitable for certain applications, it should be understood that a transceiver with both transmission and receipt capability may also be suitable for other applications, for example where communications initiated from the Smart phone could enable certain functionality of the EM.

### LED:

A light emitting diode 610 is configured to provide some feedback to the user about the operation of the EM. Although in one embodiment the operation of the EM, in terms of the integration of the Metered Dose Inhaler would be not observable, feedback is considered as valuable for a number of purposes. In one embodiment the LED would be behind the label overwrap 600 used to connect the TMAI/EM sub assembly to the MDI canister. The labels may include polymer labels that have the appropriate strength and durability characteristics to suitably maintain connection of the devices during use, but are also translucent and may permit light from an LED to be seen by the user through the label. As such, while no further customization of the integrated device is required, a convenient and low cost means of communicating information to the user is provided. The information that may be communicated may include confirmation that an actuation has been recorded, confirmation of operation, confirmation of communication or connection with a smart phone, and trouble-shooting diagnostics information in the case there is a problem. Other information about the operation of the EM, including information that the EM is detecting, storing and communicating, may also be considered. For example, the EM, via the LED, may assist the user in locating their inhaler when it is misplaced or when it is dark.

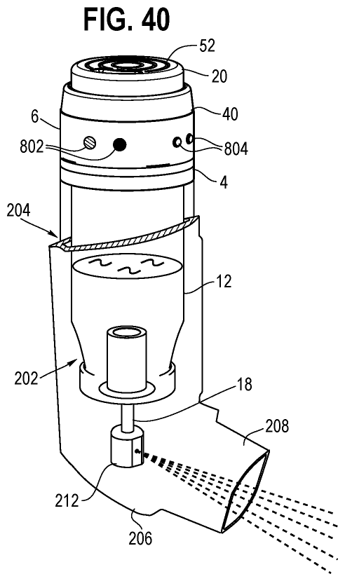

Referring to FIG. 40, the EM or eTMAI may be configured with various feedback devices and system, including for example indicator lights 802 (e.g., red and/or green LED's), that may be programmed to illuminate in response to various inputs. In an alternative embodiment, the outer casing may be made of glow in the dark materials to help user find the inhaler in low light situations. Alternatively, the device may include an ambient light sensor, such that when it is a low light, or no light, the eTMAI would periodically pulse an LED to indicate location such that the user may locate the device in a dark room. For example, the system may provide a pulse every 3 to 5 seconds, or the frequency may be programmable by the user depending on preference.

### On/Off Switch:

A switch 612 is provided that allows for the module to be turned off to conserve power. Other power control systems may be suitable, for example by remote actuation, to put the device to sleep or awaken the device, for example using an accelerometer.

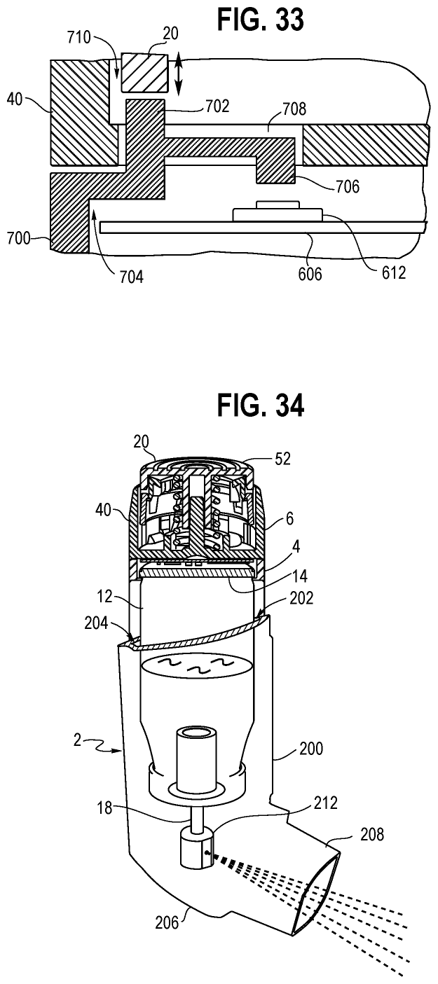

Referring to FIG. 33, one embodiment of a waking up device may accommodate a first time use only at the priming stage, and waking up the device from deep sleep by using a tactile switch 612. Counting thereafter is performed using the same switch 612. In one suitable embodiment, the tactile switch 612 may be a TL3780 Ultra Miniature. The switch is configured in normally open (“NO”) configuration. A PCBA 606 is mounted on the underside of the TMAI. A cantilever arm 700 is molded as part of the eTMAI carrier component, contoured to the cylindrical shape. The arm functions as lever, which pivots about a fulcrum 704 defined at the main junction to the base. A force is applied by the cap 20 rim to an engagement pad 702 on the cantilever spaced from the fulcrum 704, with an opposite end of the arm 706 engaging the switch positioned on the surface of the PCBA facing the base. The switch 612 is shaped and dimensioned to fit in an enlarged cutout 708 defined by the base near an orientation paddle. The engagement pad 702 of the cantilever arm extends upwardly into an interior space 710 of the TMAI to engage with the rim of the cap. Any over travel of the cap 20 is accommodated by compliance in the bending of the cantilever arm 700. The initial closing of the switch during a first priming shot may bring the processor out of a deep sleep state. Thereafter, the actuation of the TMAI is counted by this same switch.

### Alternative Power Consumption Minimization/Elimination Systems:

The various embodiments of the eTMAI may use two different power states to increase the life of the battery 604. When idle, the eTMAI enters a low power state where the power consumption is low, e.g., approximately 3.6 μA. In this state, only the accelerometer is active. When the accelerometer detects motion, it triggers the eTMAI to switch to a normal operating state where the power consumption may range between 38-286 μA. These two power states may be sufficient for most operations. However, in some embodiments, it is desirable for the battery 604 to function for up to 4 years after manufacturing, including for example up to 3 years in storage and up to 1 year of use. To meet this requirement, it is desirable to reduce power consumption while in storage. To accomplish this reduction, at least the approaches may be suitable: (1) Minimize power consumption by implementing a ‘deep sleep’ state from time of manufacture to initial setup by the end user, and/or (2) Prevent power consumption by removing contact with the battery until initial setup by the end user.

### Minimizing Power Consumption:

Under the first approach, i.e., minimizing eTMAI power consumption during storage, the device or etMAI assembly may include a 3^{rd} power state called ‘deep sleep’ where the power consumption is minimal, e.g., approximately 0.4 μA. The eTAMI will enter the deep sleep power state at time of manufacture and remain in deep sleep until an initial setup by the end user. While in the deep sleep state, the MCU on the PCBA 606 is operating with very little function and all other components such as the accelerometer and IR detection are not active. In other words, there are no inputs to signal the MCU to wake from deep sleep. Rather, various eTMAI embodiments may be configured with an internal switch that opens or closes during the first priming actuation and triggers a wake-up response in the MCU. The basic principle is described below in Table 1.

TABLE 1
Deep Sleep Wake Up Process

| Step | Action | Device Response |
|---|---|---|
| 1 | Battery is installed | Device enters deep sleep state (0.4 μA) |
| 2 | Device is in storage | none |
| 3a | User primes MDI | Internal switch momentarily closes or |
| | for the first time | opens |
| 3b | | Transistor is “shorted” to ground (0 V) |
| 3c | | MCU GPIO flips from high to low |
| | | triggering wake-up |
| 3d | | Device enters normal operating state |

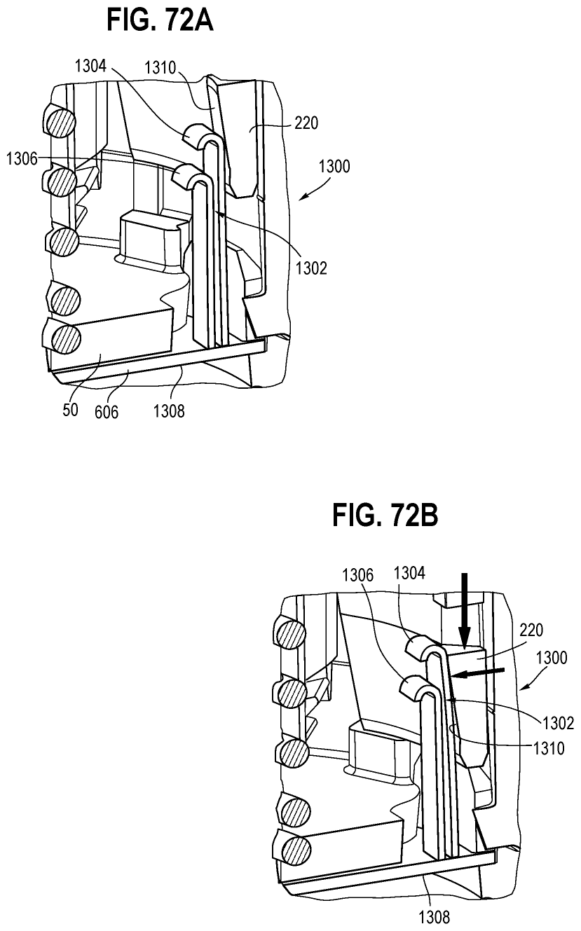

As shown in FIGS. 72A to 74B, various embodiments of a contact mechanism 1300 may be disposed between the battery 604, which is electrically connectable to the printed circuit board assembly, and the printed circuit board assembly 606. The contact mechanism 1300 is moveable between a first position wherein the battery 604 is not electrically coupled to the printed circuit board assembly 606, and the MCU in particular, and a second position wherein the battery 604 is electrically coupled to the printed circuit board 606, and the MCU in particular.

In one embodiment, shown in FIGS. 72A and B, the contact mechanism 1300 includes a switch 1302, wherein the switch 1302 is moveable between the first position where the battery is not electrically coupled of the printed circuit board, and the second position, wherein the battery is electrically coupled to the printed circuit board, in response to the movement of the cap member 220 relative to the base member 40 between the first and second positions. In one embodiment, the switch 1302 includes a pair of electrically conductive spring fingers 1304, 1306. The cap member 220 and/or the base 40 biases one of the spring fingers 1304 into contact with the other of the spring fingers 1306 as the cap member 220 is moved relative to the base between the first and second positions. Spring finger 1304 is slightly longer than spring finger 1306 in one embodiment. The spring fingers 1304, 1306 may remain in contact after a first movement of the cap relative to the base between the first and second positions.

It should be understood that in one embodiment, the switch 1302 is normally open and closing the switch initiates the wake-up process, but the opposite may also work. Consideration must also be given to any increase in force due to the energy expended bending/moving the spring finger 1306 by way of bending and friction. In one embodiment, no increase in force is experienced. In other embodiments, the increase in force is only experienced during a first priming shot.

The pair of conductive spring-fingers 1304, 1306 protrude through an opening 1308 the bottom 50 of the eTMAI base and into a cavity defined inside the base member. Each spring finger 1304, 1306 acts as a pole in the switch 1302, so the switch is normally open as the fingers are spaced apart. During compression or movement of the mechanical counter, e.g., cap and/or base, and in one embodiment skirt of the cap 220, engages one of the conductive spring-fingers 1304 and deflects the spring finger 1304 towards the other spring-finger 1306. It should be understood that the cap and base move toward each other, or relative to each other. As such, in an alternative embodiment, the base may engage and deflect one of the spring fingers coupled to the cap. When contact is made, the switch 1302 is momentarily closed, triggering wake-up. The increase to the force to actuate due to the radial force component that deflects the outer spring finger 1304, and any attendant friction, may be minimized by reducing the angle of a wedge feature, or engagement surface 1310 (e.g., inner surface of the skirt), on the cap, thereby reducing the magnitude of displacement. However, even after optimization, a small increase in the force required to actuate the dose counter may be experienced on every actuation. In one embodiment, the outer spring finger 1304 may be snapped closed, or moved over center, in response to a small radial force such that the spring fingers 1304, 1306 remain in contact for all subsequent actuations. As such, in this embodiment, there is only an increase in actuation force during a first priming shot, which does not adversely affect the dispensing of the medicament to the user.

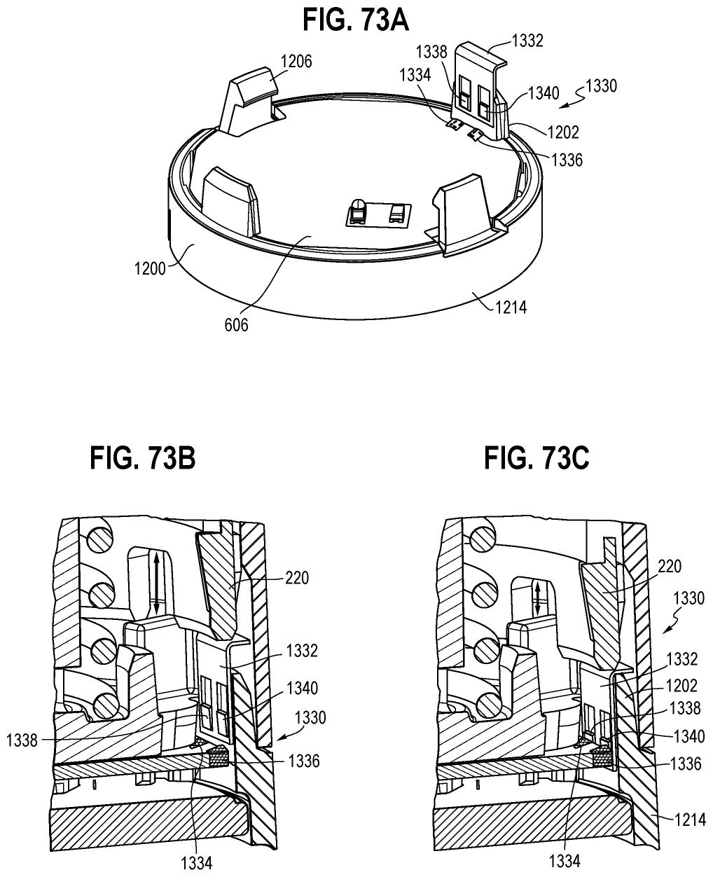

In another embodiment, shown in FIGS. 73A-C, a wake-up signal is triggered only on the first priming shot, so that the force to actuate for remaining actuations is not changed. In this embodiment, the contact mechanism 1330 includes a slide member 1332, wherein the cap and/or the base moves the slide member 1332 to close a circuit between the battery and the printed circuit board, and MCU in particular, as the cap moves relative to the base between the first and second positions. The slide member 1332 may be configured as a conductive, metal slider clipped onto the retaining ring 1214, for example by securing it to one of the tab members 1202 configured without a catch, in such a way that the slide member may only translate up and down as shown in FIGS. 73B and C. Below the slide member are a pair of electrical contact points 1334, 1336, which may be engaged by a pair of corresponding contacts 1338, 1340 on the slide member 1332, shown as wedges, on the slide member to complete the circuit and trigger the device to wake from deep sleep. In this embodiment, the slide member 1332 is designed to close the circuit when it is pushed down, but the slide member may also be pushed upwardly by the base member.

Once installed onto a TMAI, the slide member 1332 is positioned so that when the cap member 220 is pressed down, the cap member forces the slide member 132 down into contact with the PCB and closes the circuit. Once the slide member 1332 is pressed into position, the slide member remains in position by way of friction, compression, or mechanical retention, and does not thereafter increase the amount of force required to actuate the TMAI. For example, a bottom edge of the slide member 1332 may be wedged between a peripheral edge of the PCBA and the side of the base member 50 as shown in FIG. 73C.

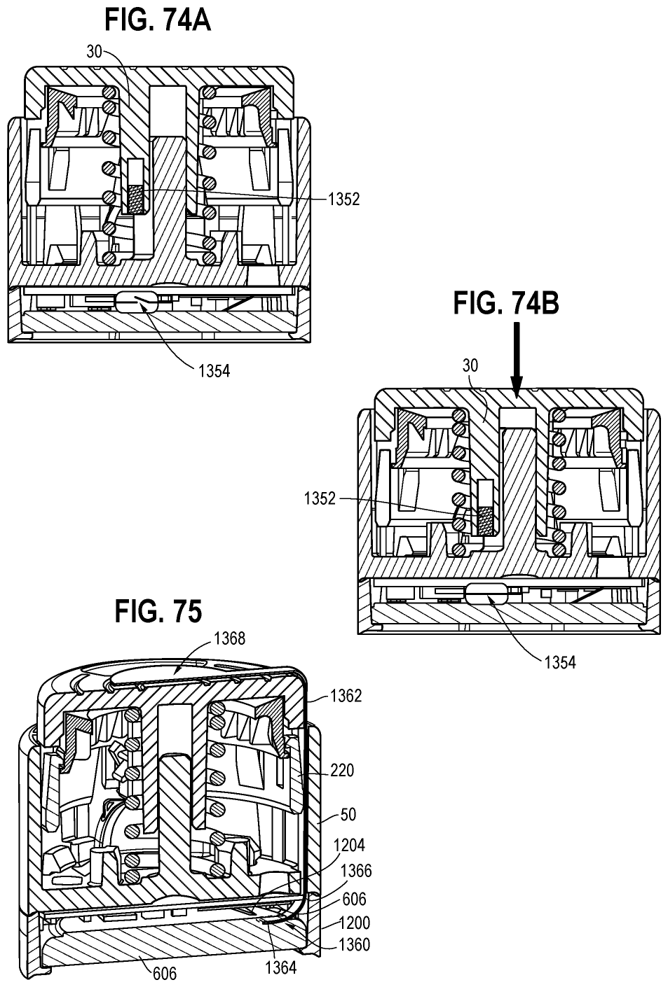

In another embodiment, shown in FIGS. 74A and B, the contact mechanism 1350 includes a switch configured as a reed switch 1354. At least one of the cap or base includes a magnet 1352, which closes the reed switch as the cap 220 moves relative to the base 50 between the first and second positions. The reed switch 1364 may be closed (or opened) by the magnet 1352 to trigger the eTMAI wake up. In one embodiment, the reed switch 1354 is disposed or located on the PCBA 606 in-line with a magnet 1352 that is embedded in the cap member 220, for example in the hub portion 30. As the cap and base are moved relative to each other, the magnet 1352 is moved into proximal relationship to the reed switch 1354, thereby causing the reed switch 1354 to close. Terminals inside the reed switch 1354 are forced closed due to magnetic forces and the circuit is closed waking up the eTMAI. One benefit of this embodiment is that the movement of the cap relative to the base is not affected by any increased friction or bending forces, and the magnetic attraction has a negligible effect on the force to actuate.

### Preventing Power Consumption:

Referring to FIGS. 75-78B, various embodiments are configured to prevent electrical contact with the battery 604 during storage and shipping and rely on the end user to ‘activate’ the device. In these embodiments, there is no deep sleep state because the battery is not electrically connected to, or powering the device until the initial setup step. Once the device is activated and the battery 604 powers up the PCBA and MCU, the device enters its normal operating state.

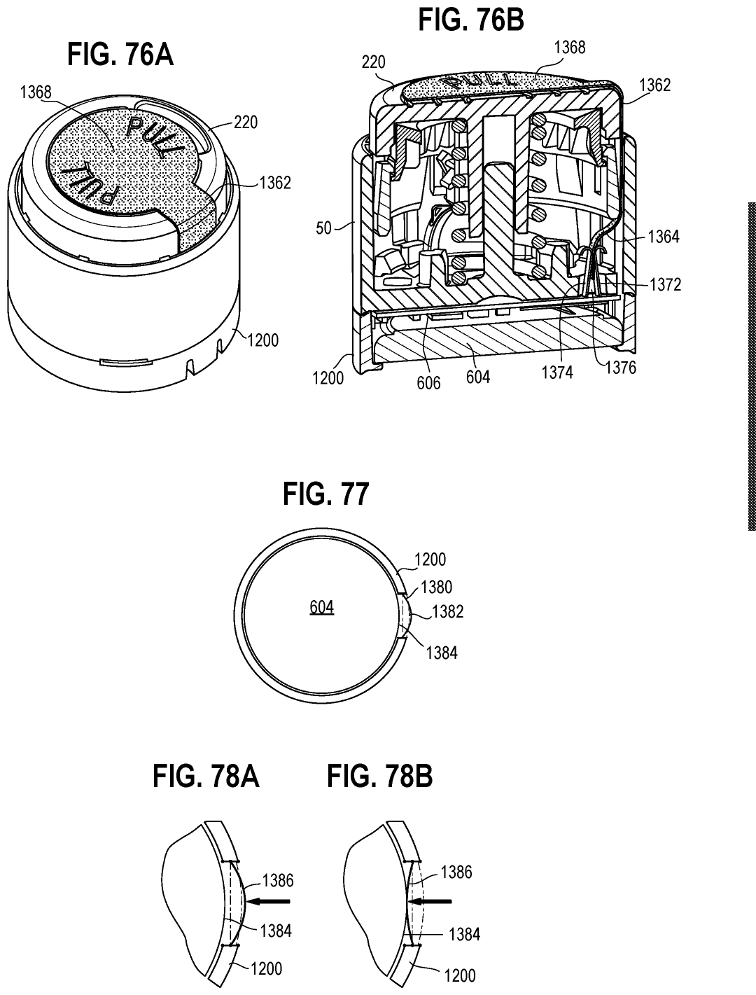

In one embodiment, shown in FIG. 75, the contact mechanism 1360 includes an insulating pull tab 1362 that prevents the battery 604 from discharging during shipping. In order to provide access to the pull tab, even when the module is secured to the TMAI with a label or wrap, the insulating pull tab is fed between the cap member 220 and the base 50 through a hole 1366 in the floor 50 of the base, and between the PCBA 606 and battery 604. A portion 1364 of the pull tab 1362 may be folded under the PCBA before the battery is installed so as to thereby prevent electrical contact with the battery. A portion 1368, or opposite end, of the pull tab has a larger profile, and is greppable, so it can be easily gripped. In one embodiment, the portion 1368 is positioned exterior to the eTMAI, and may include an adhesive, so the enlarged portion 1368 lays flat against, and is adhered to, the top of the eTMAI, e.g., cap member 220 as shown in FIGS. 76A and B. In this way, the pull tab 1362 includes a first portion 1364 disposed between a pair of contacts, and a second portion 1368 disposed exteriorly to the mechanical dose counter. In this embodiment, the pair of contacts includes the battery 604 and a contact 1252 on the PCBA.

An alternate embodiment, shown in FIGS. 76A and B, the contact mechanism 1370 includes a pair of normally closed electrical contacts 1372, 1374, configured as a pair of spring fingers, protrude through a cutout or opening 1376 in the base or floor 50 of the TMAI. During assembly, the first portion 1364 of the insulation pull tab 1362 is positioned between the contacts 1372, 1374 preventing power from discharging from the battery 604. Once the pull tab 1362 is removed, the contacts 1372, 1374 close, and power is made available from the battery. The pull tab 1362 includes a second portion 1368 positioned exterior to the eTMAI, for example being adhered to the top of the cap member.

In another embodiment, shown in FIGS. 77-78B, the contact mechanism 1380 includes a snap member 1382 moveable between the first and second positions. The snap member 1382 may be configured as an arced flexible beam 1386 moveable overcenter between the first and second positions (FIGS. 78A and B). In this embodiment, a positive or side battery terminal is not in contact with the battery 606 until the user squeezes the sides of the device, forcing the metal terminal, or beam 1386, to snap past centerline, or over center, against the side 1384 of the battery 604, with the terminal thereafter remaining in that position.

### Description of Principles of Operation:

The EM may be integrated with existing mechanical TMAI designs and their manufacturing processes. In one embodiment, the EM may be added as a simple subassembly step with straightforward attachment means. What enables this is that fact that the EM detects the actuation event via a force sensor. Similarly, the mechanical TMAI is essentially a mechanical force sensor. When a predetermined force is applied, the mechanical TMAI advances and registers that an actuation of the MDI has occurred. Similarly, the EM detects an actuation when a predetermined force has been applied, only in this case it uses electronic means for detection. By using the same methodology, it allows the two devices to be “stacked,” or arranged serially, which allows for a simplified integration.

In operation, the applied force generated by the user's finger is applied to the top of the TMAI which is then directed through the TMAI, through the EM, and then to the MDI Canister, or container. It is important to note that in this stack, i.e., serial arrangement, direct communication of forces into each component ensures stability of the mechanical contact between the EM and both the TMAI and MDI canister. This will provide precise and consistent force transfer and therefore reliable detection of the force event by the EM.

One component that to consider in this configuration is the label wrap 600 that is intended to connect the TMAI-EM assembly to the canister. Although the stack of the TMAI-EM and MDI canister will have negligible compression, the assembly should ensure that the label wrap does not introduce variability to the ability of the EM to detect a force event. The FSR used in this embodiment is selected not only for cost and integration benefits, but also because it has negligible compression. Label materials in general are highly compliant and as such, even when the label is applied, it will not appreciably interfere with the ability of the force sensor to detect the force event.

Additionally, the connection system between the EM and the TMAI should be configured to ensure proper force transfer and registration. Forces must be allowed to transfer cleanly from the users finger, through the TMAI, through the EM, and then to the canister. In a preferred embodiment, the bottom of the TMAI is modified to include a cup adapter into which the EM is pressed. Snap fits may be used to capture the EM but other methods, as disclosed above, may be used including adhesives, tapes, etc. The extension on the bottom of the TMAI may provide extended surface area to which the label could be applied which ensures a seamless integration with the MDI canister and minimal gaps which could cause creases which would be visible to the user. However, in one embodiment, the extension does not come into contact with the top of the MDI canister so as to ensure there is no force transfer between the TMAI directly to the canister, but rather is directed through the EM so as to avoid any bypass of the EM and thereby prevent the EM from detecting the force event. In one embodiment, a skirt extends down from the top of the TMAI but a gap remains between the TMAI skirt and the MDI canister in the final assembled state with the EM. This would ensure that the forces are directed from the TMAI, through the EM, and into the MDI canister and not through the skirt extension, as shown in FIG. 6.

In an alternate embodiment, the arrangement may be inverted and a separate adapter component could be used to house the EM as shown in FIG. 7. In this embodiment the stack would be such that this adapter would directly contact the top of the MDI canister and the EM would sit inside it. The TMAI would then sit directly on top of the EM. In this embodiment, a gap is required between the adapter and the TMAI, similar to the gap described above with respect to the embodiment of FIG. 6. Various connection systems may then be used to couple the EM to the TMAI, for example with snap fits, adhesives etc., as shown in FIG. 7.

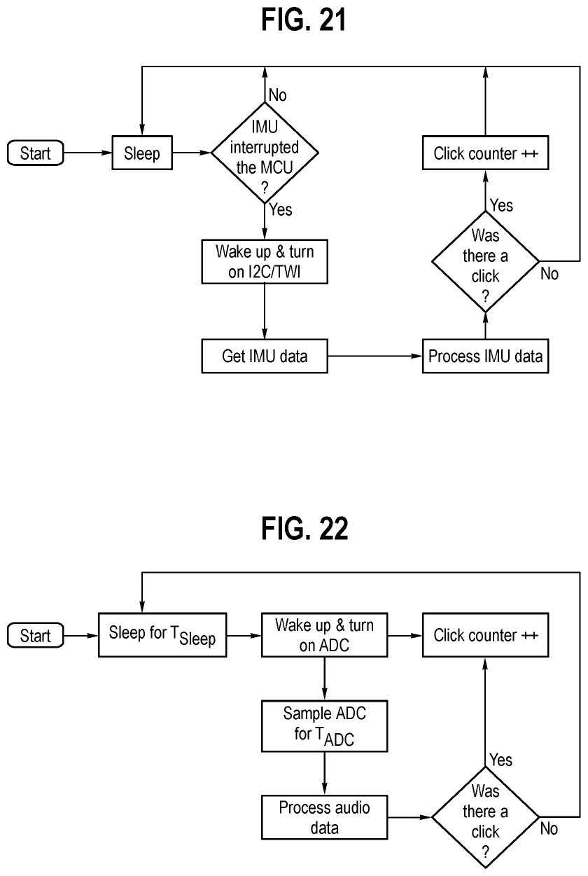

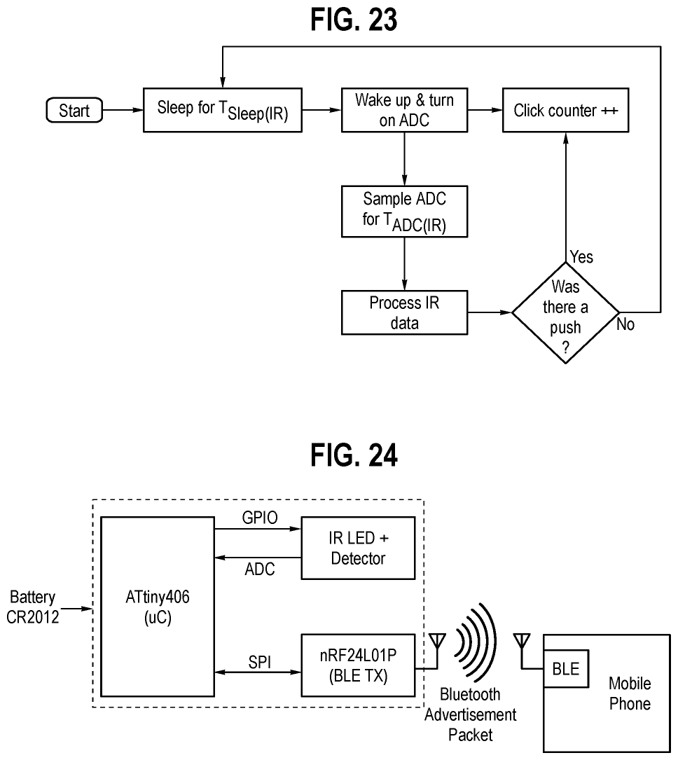

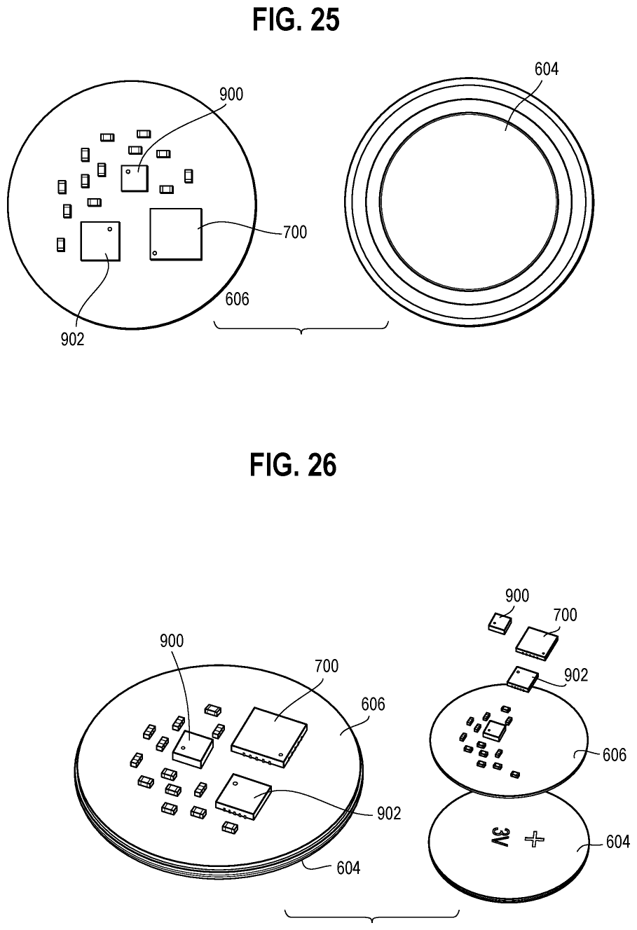

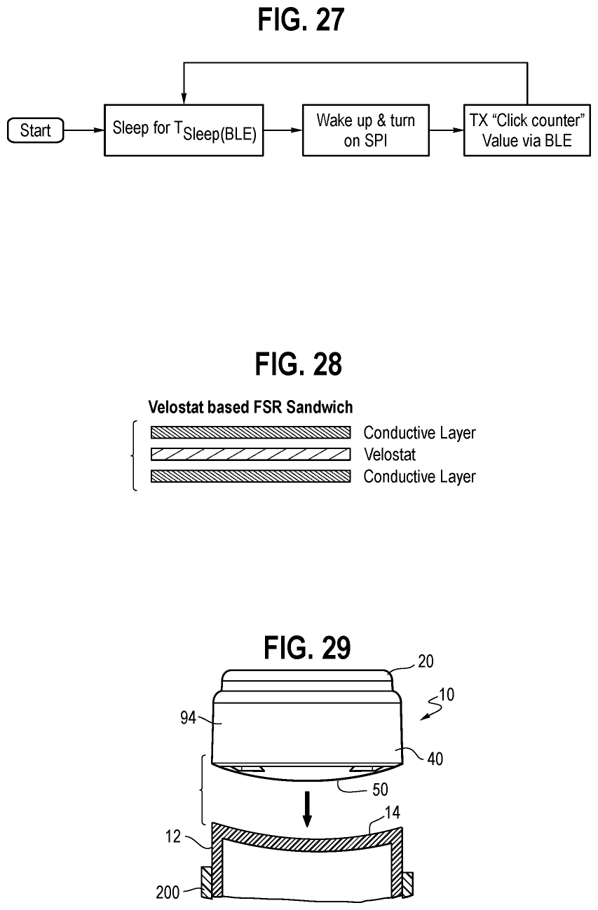

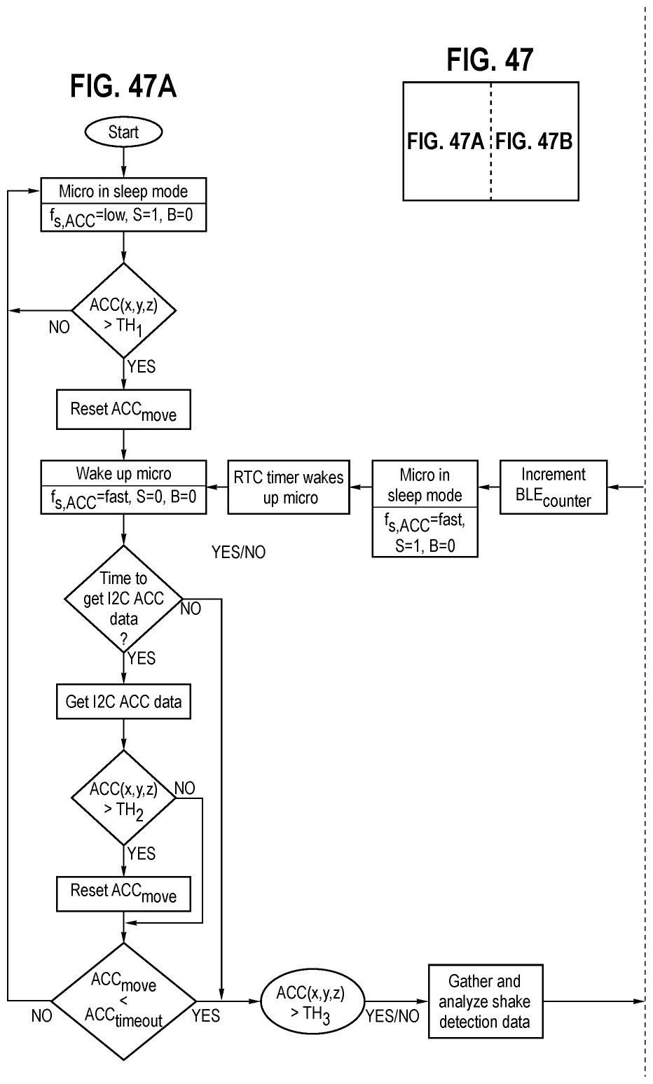

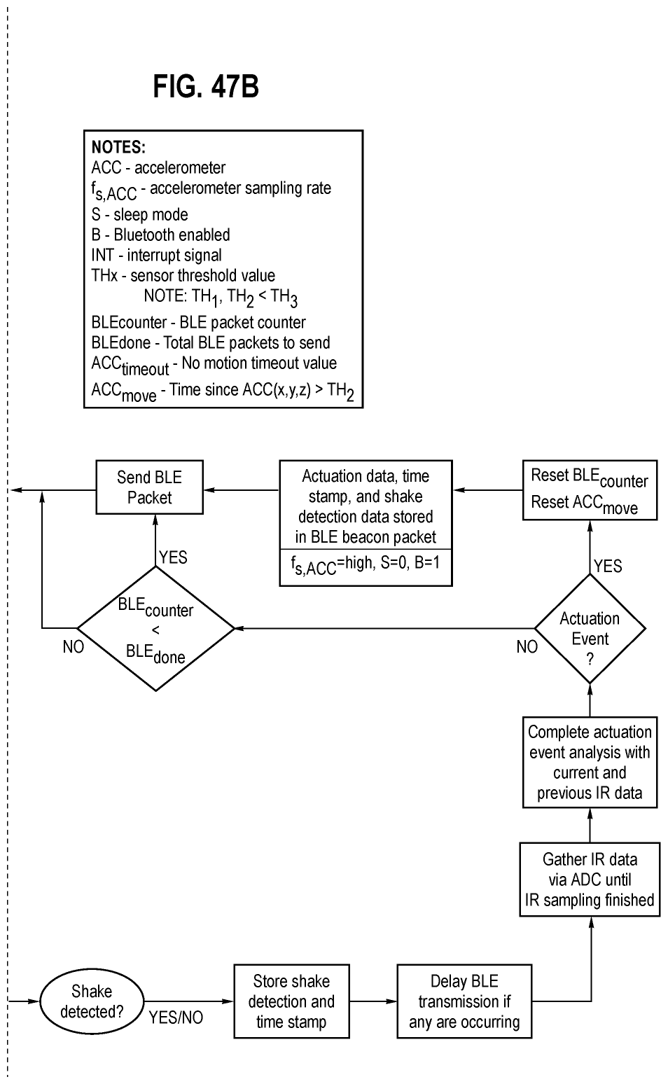

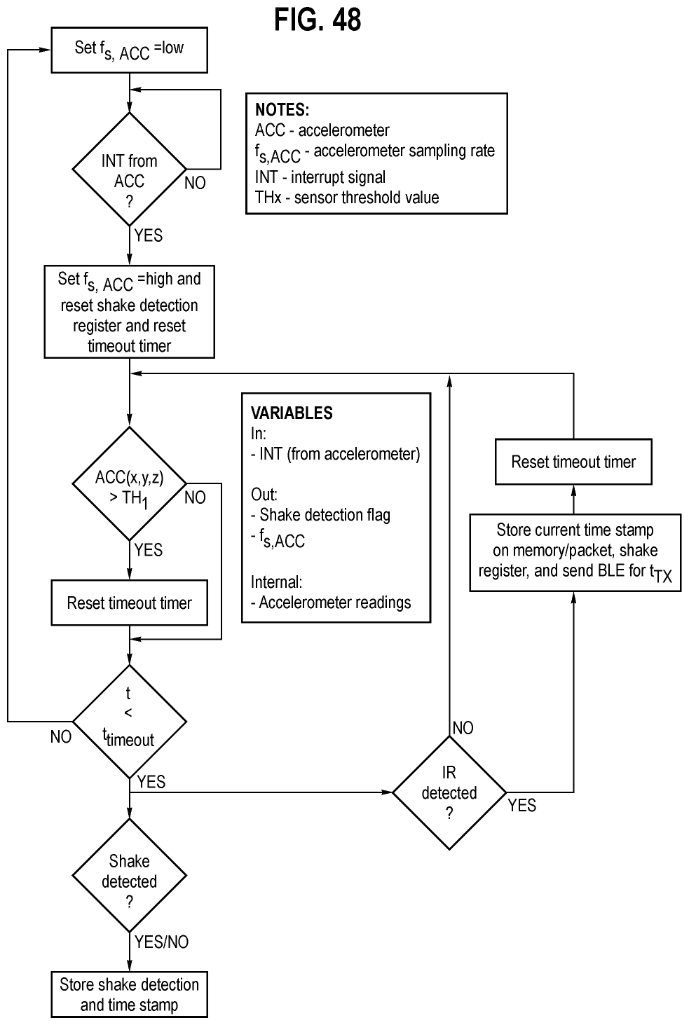

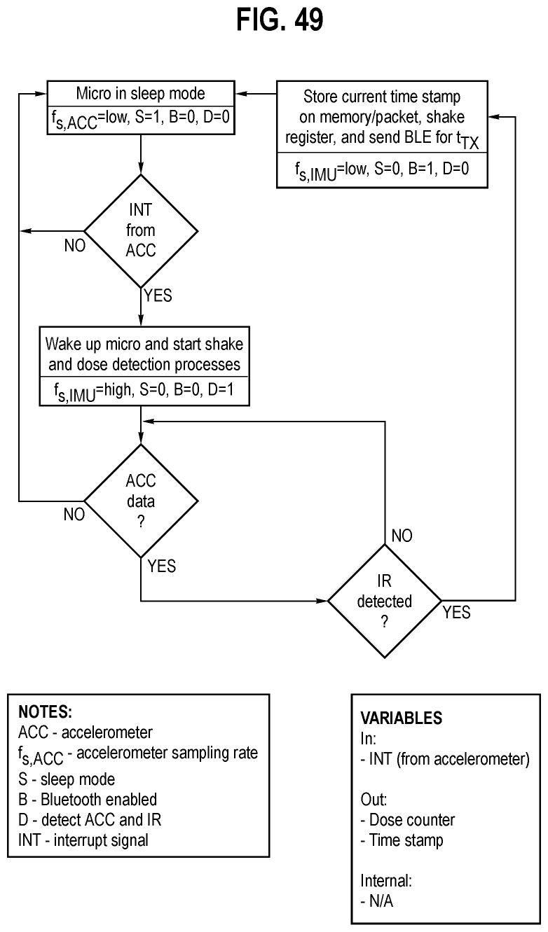

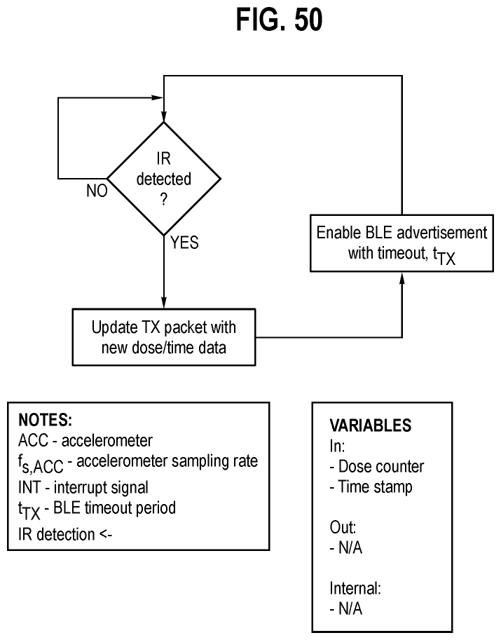

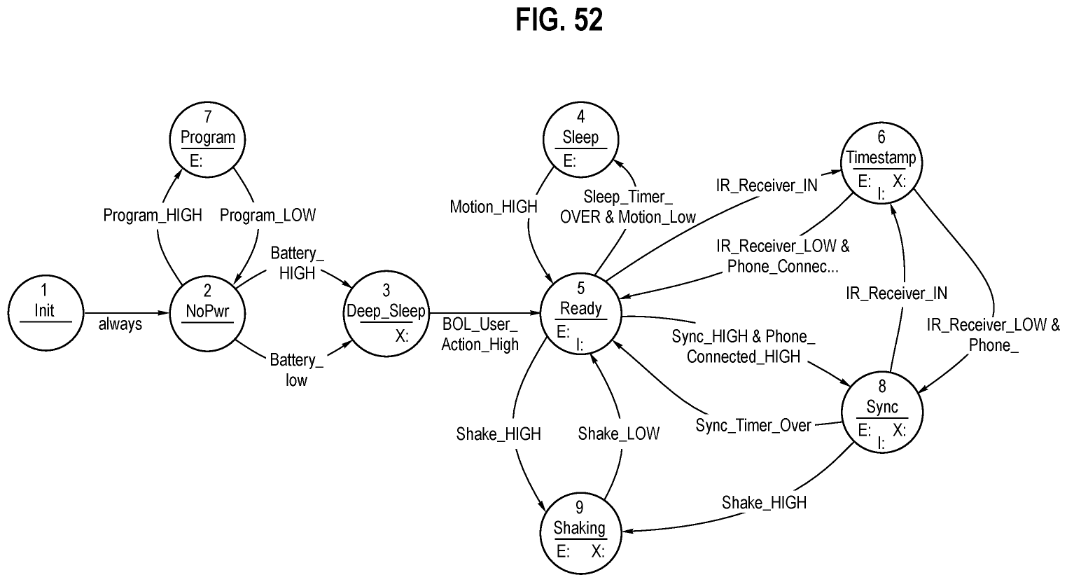

In operation, and referring to FIGS. 47-52, the EM would electronically register each actuation once a predetermined force is applied to the force sensor. A microprocessor would monitor the force response of the force sensor and determine when an actuation has occurred. When an actuation is registered it will be stored into memory. When connected to a smart phone, packets of information representing, at a minimum, the actuation registered and its corresponding time and date would be sent. The EM could also have a decrement counter that continually updates and subtracts actuations so that an accurate “doses” or “counts” remaining is calculated. This information can also be sent to the connected device. Referring to FIGS. 21-23, 27 and 47-52, the operation of the device and system is shown.

### Other Alternate Embodiments:

### Alternate Locations for EM:

The EM may operate as a dose counter or tracker in many different configurations, with or without being integrated with the TMAI. In one embodiment, shown in FIG. 8, the EM 4 may be configured to be integrated with the top of the TMAI instead of the bottom. In this configuration, the users finger would contact the top of the EM instead of the TMAI and thus the actuation force would be directed through it into the top of the TMAI. There are other mechanical considerations with this embodiment that would need to be considered including that the EM defines the user interface and must therefore be suitable to be touched by the users finger. In addition, any mechanical counting displays on the top of the TMAI would have to be visible and so not blocked or hindered by the addition of the EM, for example by providing a viewing window in a side wall of the TMAI, with an indicator and indicia visible therethrough.

In another embodiment, the EM may be a standalone dose counter or tracker on its own, exclusive of any mechanical device. The low cost and slim integration that make the EM suitable for integration with a mechanical dose counter is also advantageous in a standalone configuration. In this embodiment, the EM may or may not add a display element which would communicate directly to the user the number of actuations remaining, as do mechanical dose counters. When configured without a display element, the EM may instead communicate the dose or count status and tracking information through a smart phone. Integration of the EM to the top of the MDI canister may be required so that, like the earlier embodiment, it forms a suitable user interface for the user's finger. Attachment of the EM to the MDI canister may be achieved in a number of different ways including via adhesives or the addition of an adapter component that will house the EM and also facilitate attachment to the MDI canister, for example a friction fit collar or combination with other attachment means, as shown in FIG. 9.

In another embodiment, the EM may be attached or integrated with the actuator portion of the MDI, or a bottom of the actuator housing. As a low cost force based counter/tracker, the EM may be well suited to being added in this configuration as it would require very little modification to the existing and well proven MDI platform. Like the earlier embodiment where it is attached to the MDI canister, in this configuration the EM may also have a display to communicate doses remaining or tracking information but similarly may not incorporate a display or screen, but instead rely on the screen of the smart phone or connected device. Attachment to the bottom of the MDI actuator may be carried out in a number of standard ways including snap fit, press fit, adhesive, etc. As in other embodiments, the bottom of the EM would become part of the user interface where the use applies the actuation force.

### Alternate Embodiments—Alternate Count Detection Method—Infrared Sensor Displacement Sensor

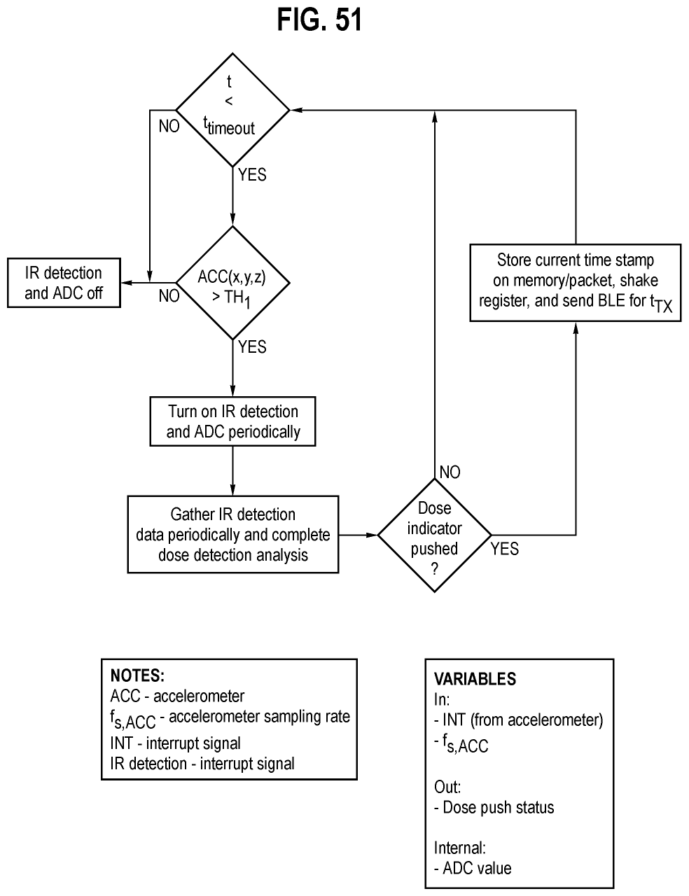

Like the force sensor described in the preferred embodiment above, whatever actuation detection method used preferably has low power consumption, low cost, and very small overall packaging. An alternate method that satisfies these requirements incorporates an Infrared LED 620 and sensor 622 to detect the displacement of the internal TMAI components. Although the TMAI is a force based counter, it still requires displacement to actuate and achieves a very accurate and consistent displacement to actuate. In one embodiment, the infrared LED and sensor would reside on the EM where the EM would have a similar overall shape and size to the one disclosed above with a force sensor. In this embodiment however, instead of a force-based methodology for detecting actuation events, it would depend on displacement of the TMAI mechanism, i.e., include a displacement sensor. In this embodiment, the EM would be similarly situated between the bottom of the TMAI and the top of the MDI canister. An infrared LED and sensor would be configured to be directed up and inside the TMAI mechanism where the infrared LED 620 could illuminate features within the TMAI mechanism. The LED light would shine vertically up into the TMAI and the sensor 622 would sense or read the light bouncing back as shown in FIG. 11, with the operation disclosed in FIG. 51. The displacement sensor may also be disposed between a base and cap of the TMAI. It should be understood that other types of displacement sensors besides the infrared LED and sensor may be suitable, for example various proximity switches.

Conveniently, the bottom of the TMAI may already have a number of holes or openings 624 to allow for manufacturing and molding. These holes or openings 624, or new holes, may be used to allow the LED and sensor to access the internal mechanism and detect the movement of one or more components of the mechanism. Detection may be done by sensing the movement of existing features within the TMAI mechanism which may including any of the mechanical elements (cap, gears, supports, guiding features, etc.). Alternately, with minimal modification to the existing TMAI mechanism, the existing features inside the TMAI can be modified to enhance the ability and precision of the infrared sensor displacement detection. This may include optimizing the position and shape of the features as well as the colour or texture. Additionally, a feature may be added, for example a post with a flat top, that would have no impact on the TMAI mechanism but would optimize displacement detection of the infrared sensor and bring the mechanical features into closer range of the sensor. Other factors may also be identified for optimization with minimal effect on the basic TMAI mechanism. This may include ensuring that minimal ambient light intrusion occurs that may interfere with the ability of the infrared sensor to pick up the actuation event. The infrared sensor approach may offer some advantages over force sensing as it eliminates any integration with the MDI canister as mentioned above. All required interfaces to enable correct operation can be contained with the TMAI and EM. In operation, the EM would be programmed with a displacement that once exceeded by the TMAI, would register an actuation. For example, in one embodiment, the TMIA has approximately a 3.5 mm total travel from a nominal, at-rest position to being bottomed out at a maximum depressed position. The actuation point typically occurs about midway through the total travel. Alternate Embodiments—Alternate Count Detection Method—Infrared Sensor Displacement Switch

The eTMAI and/or EM is configured such that it may provide various count detection methods and features, including; (1) Sound of aerosol “woosh” release from the canister picked up by the microphone inside eTMAI, recording an actuation count; (2) Flow of air in the airflow communicating channel picked up by the flow sensor, recording and actuation count; (3) Drop in pressure in the air flow communicating channel picked up by the pressure sensor, recording an actuation count; (4) Temperature sensor inside the eTMAI would take the temperature of the canister, which is much colder when actuated, it would indicate device was used, therefore record or verify an actuation count; (5) Microphone in the eTMAI picking up the signature click of the actuation rotating gears of a mechanical dose counter such as TMI dose counter etc.; and/or combinations of the various device and systems disclosed herein, which may improve the overall veracity of the system by verifying counts and thereby increasing the accuracy of the data captured and/or reported.

### Alternate Embodiments—Alternate Count Detection Method—Infrared Sensor Displacement Switch

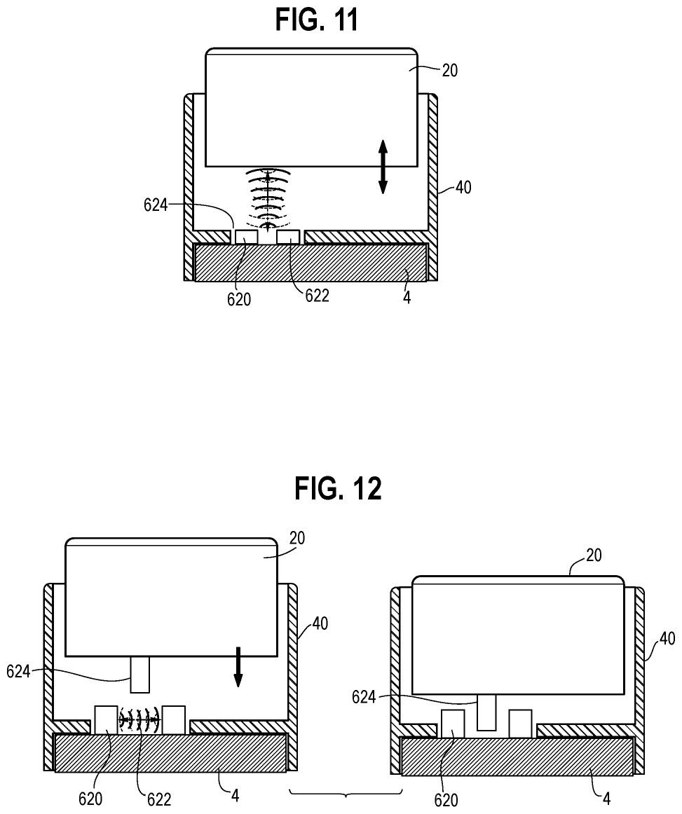

An alternative displacement sensor includes many of the same features as the displacement sensor disclosed above that incorporates an infrared LED and sensor, but instead of measuring the amount of displacement and determining whether an actuation event has occurred when a predetermined displacement has been reached, the alternate embodiment may be used to configure the infrared LED light to be interrupted fully by a feature from the TMAI. In this configuration, which also is configured as a displacement sensor as shown in FIG. 12, the infrared sensor and detector would be arranged in a configuration where they were opposite each other. The LED would shine directly at the sensor. Actuation would be determined when a blocking feature 624 from the moving part of the TMAI breaks the light beam which would be detected by the sensor and determined by the CPU to be an actuation, or a measurement of a predetermined displacement has been satisfied. In this embodiment, which is directed to whether a threshold displacement has been reached, rather than measuring the displacement, the system does not need to detect specific displacements. In this way, the displacement sensor functions as more of a switch configuration (or absolute displacement), with the difference between not-actuated and actuated, in terms of sensed IR energy could be made to be quite significant and therefore may be more tolerant to sources of interference including outside light emissions. The blocking feature 624 is incorporated into the TMAI that is configured to break the light beam from the LED at a predetermined displacement that corresponds to the actuation point of the TMAI. Since the actuation point of the TMAI occurs at the mid-point of its total travel, provision for over travel of the added beam interrupter feature would have to be provided.

### Other Alternative Sensors

Once an actuation is detected and stored by the EM, and referring to FIGS. 47-51, the EM may wirelessly transmit the time and day of the actuation(s) and the total number of button presses that occurred to the user's mobile device app.

Besides the force and displacement sensors disclosed above, other basic types of sensors, or combinations thereof, may also be suitable, with each sensor measuring one or more of the following: force, motion, sound, or distance to detect an actuation, e.g., a button press (downward push). For example, the motion may be sensed by an IMU sensor where it will detect the vibrations generated by a user button push as well as by the spring-like mechanical component that decrements the mechanical counter. Since the button push generates a clicking sound, this noise can be sensed by a MEMS microphone. In addition, the distance of the mechanical actuation, e.g., button push, may be detected by an IR emitter (LED) and detector (phototransistor) where it behaves like an IR proximity sensor, or displacement sensor, as described above.

In various embodiments, the eTMAI or EM may be configured such that provides the various sensing functions and features: (1) inhalation detection: by sound, flow, or pressure sensor, confirming the user inhaled correctly during the administration of the drug; (2) Inhaler Identification: identifying the type of drug being used, assembled at Pharma, programmed to drug type, including a module attached to canister and paired to the pMDI actuator, with the EM communicating with the pMDI actuator or vice versa; (3) Shake Detection: Accelerometer 900 shake detection sensor, monitors for shake event and/or effectiveness of shake for proper mixing of drug before use; (4) Actuation Detection via Canister Temperature: Temperature of canister would drop when actuated due to rapid expansion of propellants, indicating a device was used/triggered; (5) Actuation Detection via Sound: Microphone mounted to or near the canister listening to sounds from inside the canister, with the canister amplifying sound like a speaker box; (6) Actuation Detection by Chemical Sensor: Chemical/Bio-Marker smell sensor to detect type of drug actuated, and/or to detect propellant released; (7) Actuation Detection by Humidity or Moisture Sensor: Humidity sensor to detect amount of humidity at drug release, therefore confirming actuation; (8) Movement or Handling Sensing and Tracking: Information, interpreting accelerometer data, usage technique; and (9) Location: Geographic or physical location of device, connecting to an application in one embodiment.

### Detailed Embodiments of Sensing Inhalation Detection.

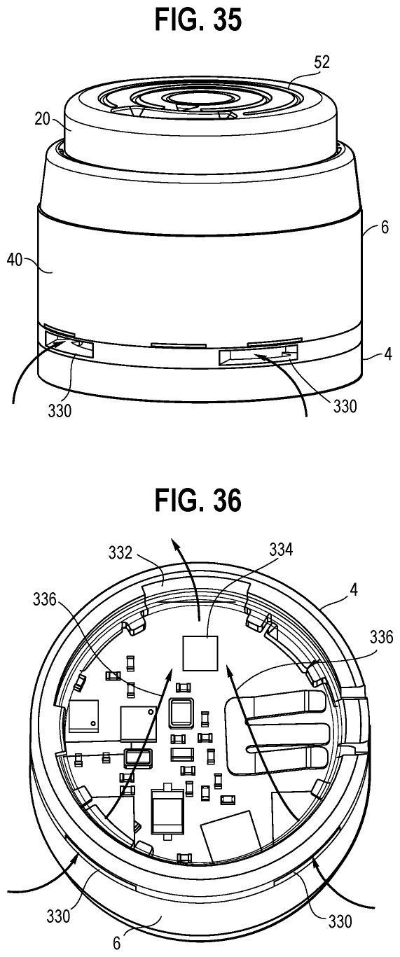

The current TMAI sits atop the MDI canister and is generally removed from the air inlet of the MDI which is formed by the gap between the pMDI canister outer wall and the inner wall of the pMDI actuator body. In one embodiment, referring to FIGS. 35-39, a plurality of air inlets 330 or channels, shown as two, are formed in the side wall of the EM housing. One or more outlets 332 are also defined in the bottom of the EM. A flow passageway 336 is defined between the inlets 330 and outlet(s) 332. One or more sensors 334 may be located on the bottom of the EM circuit board and located in the flow passageway. In this way, the EM of the eTMAI is configured to determine inhalation sensing. The top mounted dose counter EM is attached to the top of the drug canister 12. An extension, defined as a skirt 320, may be coupled to the EM to extend a plurality of air flow pathway communicating channels 338 into the interior of the MDI boot. The air flow pathway communicating channels 332 are configured to ensure air drawn by the patient during inhalation is moved through the inlet 330 and flow passageway 336 and through the outlet 332 and into the channels 338. As the eTMAI is connected to the canister, the air inlet channels must be allowed to move with respect to the pMDI actuator so that it is not impeded and so effective actuation of the pMDI can take place. As such, the air inlet channel walls can be made of two shot soft silicone rubber material or to fit the MDI shape by matching flexible plastics in a precise way that allows for gaps to be minimized while allowing translation during actuation. Importantly, a relatively leak free and movable seal may be formed to ensure a sufficient and consistent flow of air is drawing through the air inlet channels 338. This ensures that there is sufficient inhalation flow signature for the sensor to register the flow. In one embodiment, flow, pressure, and microphone sensors may be located inside the eTMAI EM. In use, air would be drawn through ports 330 located within the TMAI body, either through existing gaps in the design that would allow sufficient air flow or through dedicated ports 330. Ports would be designed in such a way as to direct a sufficient amount of air flow over the sensor 334.

Referring to FIGS. 35-38, the skirt fitting around the dose counter has a shape that fits in the MDI boot and allows for a sliding movement up and down forming the same air flow pathway communicating channel as above, which allows the air flow to communicate with the eTMAI sensors. The skirt and flow channels may direct air past one or more sensors. For example, the sensor may include a microphone to pick up changes in sound as the flow increases or decreases. Alternatively, a pressure sensor may respond to the vacuum generated during inhalation, which would provide an output that is proximal to flow. In both cases, an algorithm may be used to convert sound or pressure outputs from the sensor into air flow. The flow, sound and/or pressure sensors may be located in the EM or at some point within the flow channel or skirt. In the latter embodiment, this may allow the sensor to be positioned closer to where the source of flow is and thereby increase sensitivity and robustness of the readings. A connector wire or wireless communication may allow the EM to communicate with the sensor.

The container has a first end with a valve stem coupled to the boot, with the electronic module coupled to the opposite second end of the container. The skirt is disposed in the space between an exterior surface of the container and an interior surface of the actuator boot. The skirt extends along a side of the container. The channel 338 extends longitudinally and defines an exit port 340 at a bottom of the skirt

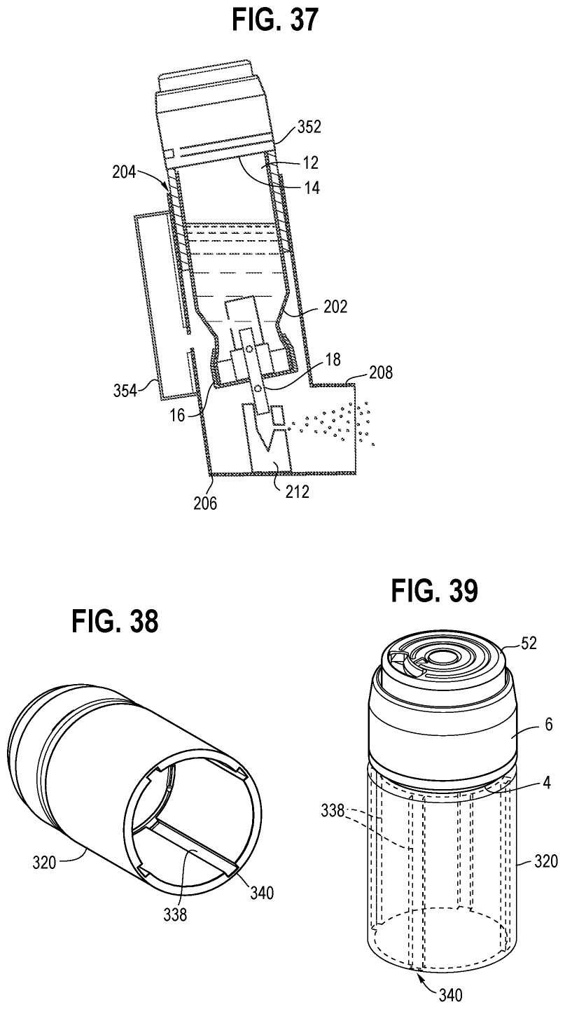

Referring to FIG. 37, in one embodiment, the eTMAI includes two units or modules 352, 354, with the module 354 being reusable and rechargeable and the module 352 being consumable or disposable. By bifurcating the modules, production costs may be reduced and also would make it easier for the user to connect to the Smart Phone App since they would not need to customize or program their device every time they buy a new boot. The programing and customization may be done once for the module 354. However, the module 352 may automatically connect to the module 354 upon installation and wake up from deep sleep. The module 354 may be coupled to the exterior of the actuator boot, for example with adhesive, and may include a larger rechargeable battery, while the smaller non reusable unit 352 may include a coin cell battery within the top mounted mechanical counter. The electronic unit 352 attached to the mechanical counter and the canister is not reusable. The two units would communicate together via very low power, with the main unit 354 then transmits a signal further to the smart phone application or other communicating systems.

In order to complete the task of detecting a user button press action, a microcontroller with one or more of a motion, sound, and/or distance sensor may be used. The sensor input data will be processed by the microcontroller, to detect if there was an actuation, e.g., a button push by the user, and then transmitted via wireless communication to a mobile app. The microcontroller does not necessarily require an embedded wireless communication capability to transmit the data to the mobile device. Instead, it may have an external wireless transceiver IC.

Some exemplary selection parameters for the microcontroller selection are listed below where it applies to both microcontrollers with and without embedded wireless communication capability.

Small Form Factor.
E.g., smaller than VFQFN-20 for microcontroller without BLE transceiver and smaller than VFQFN-48 for microcontroller with internal wireless transceiver.

Able to operate between 1.8 V to 3.6 V supply.

Consists of internal RC oscillator (both fast and slow clocks).
SoCs with embedded wireless transceiver may include external crystals.

Has SPI communication capability.
Needed for microcontrollers without embedded wireless transceiver and need to communicate to an external transceiver such as the nRF24L01+.
SPI (or I²C/TWI) may also be needed if sensors such as IMU is used.

Has ADC block.
Needed for MEMS microphone and IR detector (phototransistor).
Has internal reference voltage for the ADC.
Not required if sensors use SPI (or I²C/TWI).

Low power consumption.
E.g., less than 10 μA in sleep mode.

Has enough RAM and flash for click detection processing (to be determined).

The microcontroller may also include blue tooth. In order to reduce the overall cost of the click detector module, one embodiment includes a separate transceiver for the BLE communication.

The parameters for sensors to be used for clicking sound detection include:

Small form factor.
E.g., smaller than VFQFN-20 and height of less than 1 mm.

Able to operate between 1.8 V to 3.6 V supply.

Uses SPI or I²C communication if it uses digital communication for data output.

Low power consumption.
E.g., less than 10 μA in low-power mode (averaged for one hour operation).

Another sensor may be an Inertial Measurement Unit 900 (IMU)=gyro, accelerometer, etc. The push button TMAI device generates vibration when the button is pressed down, from both the motion of the button being pressed down as well as from the clicking mechanism for decrementing the mechanical counter. This motion can be captured by an IMU sensor 900 such as an accelerometer.

The vibration generated from an actuation, e.g., a button press, is captured by the IMU sensor 900 which then triggers the interrupt event and sends a wake-up signal to the microcontroller 902. The microcontroller 902 will wake-up from sleep mode and begin recording the IMU sensor data for T_{IMU} seconds. Once the data collection is complete, the recorded data will be processed using DSP algorithms (e.g., FFT or Goertzel algorithm) along with previously recorded profile data of a button push to determine if the button was pushed or not.

The IMU sensor may operate in low-power mode until motion is detected and outputs an event trigger signal for the microcontroller to wake-up (this trigger event occurs if one or more axis readings go above a programmed threshold value). This lowers the overall power consumption for both the IMU and the microcontroller, since the microcontroller does not have to poll continuously to check if motion was detected or not.

One suitable IMU embodiment is a KXTJ3-1057 accelerometer. The IMU sensor includes shake detection capability and consumes relatively low power during both sleep and sensor reading modes (i.e., 0.9 and 10 μA). In one embodiment, the vibrations caused by the button being pushed are due to the clicking sound generated from the spring-like mechanical components within the mechanical dose counter.

In another embodiment, the push button device consists of a mechanical counter that decrements the counter value each time the user pressed down the button. When the counter decrement occurs, a clicking sound is generated which can be captured by a microphone. The clicking sound generated from an actuation is picked up by the microphone and the audio signal is read by the microcontroller ADC. The microcontroller will read the audio data for T_{ADC} seconds. Once the data collection is complete, the recorded audio data will be processed using DSP algorithms (e.g., FFT or Goertzel algorithm) along with previously recorded profile data of a button push to determine if the button was pushed or not. After processing is complete, the microcontroller will go to sleep for Tₛₗₑₑₚ and then repeat the previous steps. In one embodiment, a digital MEMS microphone (with PDM signal output) with internal amplifier may be used.

In another embodiment, small holes are provided underneath the mechanical dose counter where the action of the cap being pressed down can be observed. That is, when the cap is pressed down, the spring-like mechanical component gets closer towards the hole. A proximity sensor, or displacement sensor, would be able to detect the distance of the mechanical component through the hole which in turn detects if the cap and mechanical dose counter has been actuated. The cap press is detected by the microcontroller with the use of a combination of IR detector and emitter. The microcontroller will read the IR detector (phototransistor) data for T_{ADC IR} seconds. Once the data collection is complete, the recorded IR intensity (distance) data will be processed to determine if the button was pushed or not. After processing is complete, the microcontroller will go to sleep for Tₛₗₑₑₚ and then repeat the previous steps.

Various microcontrollers operate only in Bluetooth advertising mode or establish a connection between the mobile device and themselves.

As shown in FIG. 26, the EM includes a battery, PCB, and components. The diameter of the entire module is 20 mm and the maximum height between the battery and the largest circuit component (located at the centre with the IC) is 2.55 mm. The height of the module, near the ends of the PCB between battery and the PCB, is 1.6 mm, which means this module can fit into our required volume assuming the indent curvature of the inhaler metal capsule exists.

In order to provide faster and more accurate processing of the sensor data generated within the EM, data may be wirelessly communicated to a smart phone, local computing device and/or remote computing device to interpret and act on the raw sensor data.

In one implementation, the EM includes circuitry for transmitting raw sensor data in real time to a local device, such as a smart phone. The smart phone may display graphics or instructions to the user and implement processing software to interpret and act on the raw data. The smart phone may include software that filters and processes the raw sensor data and outputs the relevant status information contained in the raw sensor data to a display on the smart phone. The smart phone or other local computing device may alternatively use its local resources to contact a remote database or server to retrieve processing instructions or to forward the raw sensor data for remote processing and interpretation, and to receive the processed and interpreted sensor data back from the remote server for display to the user or a caregiver that is with the user of the MDI.

In addition to simply presenting data, statistics or instructions on a display of the smart phone or other local computer in proximity of the MDI configured with an EM, proactive operations relating to the MDI may be actively managed and controlled. For example, if the smart phone or other local computer in proximity to the MDI determines that the sensor data indicates the end of treatment has been reached, the smart phone or other local computing device may communicate directly with the EM to provide a signal, such as an audio or visual signal. In yet other implementations, real-time data gathered in the EM and relayed via to the smart phone to the remote server may trigger the remote server to track down and notify a physician or supervising caregiver regarding a problem with the particular drug delivery session or a pattern that has developed over time based on past sessions for the particular user. Based on data from the one or more sensors in the EM, the remote server may generate alerts to send via text, email or other electronic communication medium to the user's physician or other caregiver.

The electronic circuitry in the EM, the local computing device and/or the remote server discussed above, may include some or all of the capabilities of a computer 500 in communication with a network 526 and/or directly with other computers. As illustrated in FIG. 30, the computer 500 may include a processor 502, a storage device 516, a display or other output device 510, an input device 512, and a network interface device 520, all connected via a bus 508. The computer may communicate with the network. The processor 502 represents a central processing unit of any type of architecture, such as a CISC (Complex Instruction Set Computing), RISC (Reduced Instruction Set Computing), VLIW (Very Long Instruction Word), or a hybrid architecture, although any appropriate processor may be used. The processor 502 executes instructions and includes that portion of the computer 500 that controls the operation of the entire computer. Although not depicted in FIG. 31, the processor 502 typically includes a control unit that organizes data and program storage in memory and transfers data and other information between the various parts of the computer 500. The processor 502 receives input data from the input device 512 and the network 526 reads and stores instructions (for example processor executable code) 524 and data in the main memory 504, such as random access memory (RAM), static memory 506, such as read only memory (ROM), and the storage device 516. The processor 502 may present data to a user via the output device 510.

Although the computer 500 is shown to contain only a single processor 502 and a single bus 508, the disclosed embodiment applies equally to computers that may have multiple processors and to computers that may have multiple busses with some or all performing different functions in different ways.

The storage device 516 represents one or more mechanisms for storing data. For example, the storage device 516 may include a computer readable medium 522 such as read-only memory (ROM), RAM, non-volatile storage media, optical storage media, flash memory devices, and/or other machine-readable media. In other embodiments, any appropriate type of storage device may be used. Although only one storage device 516 is shown, multiple storage devices and multiple types of storage devices may be present. Further, although the computer 500 is drawn to contain the storage device 516, it may be distributed across other computers, for example on a server.

The storage device 516 may include a controller (not shown) and a computer readable medium 522 having instructions 524 capable of being executed on the processor 502 to carry out the functions described above with reference to processing sensor data, displaying the sensor data or instructions based on the sensor data, controlling aspects of the smart nebulizer to alter its operation, or contacting third parties or other remotely located resources to provide update information to, or retrieve data from those remotely located resources. In another embodiment, some or all of the functions are carried out via hardware in lieu of a processor-based system. In one embodiment, the controller is a web browser, but in other embodiments the controller may be a database system, a file system, an electronic mail system, a media manager, an image manager, or may include any other functions capable of accessing data items. The storage device 516 may also contain additional software and data (not shown), which is not necessary to understand the invention.

The output device 510 is that part of the computer 500 that displays output to the user. The output device 510 may be a liquid crystal display (LCD) well-known in the art of computer hardware. In other embodiments, the output device 510 may be replaced with a gas or plasma-based flat-panel display or a traditional cathode-ray tube (CRT) display. In still other embodiments, any appropriate display device may be used. Although only one output device 510 is shown, in other embodiments any number of output devices of different types, or of the same type, may be present. In an embodiment, the output device 510 displays a user interface. The input device 512 may be a keyboard, mouse or other pointing device, trackball, touchpad, touch screen, keypad, microphone, voice recognition device, or any other appropriate mechanism for the user to input data to the computer 500 and manipulate the user interface previously discussed. Although only one input device 512 is shown, in another embodiment any number and type of input devices may be present.

The network interface device 520 provides connectivity from the computer 500 to the network 526 through any suitable communications protocol. The network interface device 520 sends and receives data items from the network 526 via a wireless or wired transceiver 514. The transceiver 514 may be a cellular frequency, radio frequency (RF), infrared (IR) or any of a number of known wireless or wired transmission systems capable of communicating with a network 526 or other smart devices 102 having some or all of the features of the example computer of FIG. 2. The bus 508 may represent one or more busses, e.g., USB, PCI, ISA (Industry Standard Architecture), X-Bus, EISA (Extended Industry Standard Architecture), or any other appropriate bus and/or bridge (also called a bus controller).

The computer 500 may be implemented using any suitable hardware and/or software, such as a personal computer or other electronic computing device. The computer 500 may be a portable computer, laptop, tablet or notebook computers, smart phones, PDAs, pocket computers, appliances, telephones, and mainframe computers are examples of other possible configurations of the computer 500. The network 526 may be any suitable network and may support any appropriate protocol suitable for communication to the computer 500. In an embodiment, the network 526 may support wireless communications. In another embodiment, the network 526 may support hard-wired communications, such as a telephone line or cable. In another embodiment, the network 526 may support the Ethernet IEEE (Institute of Electrical and Electronics Engineers) 802.3x specification. In another embodiment, the network 526 may be the Internet and may support IP (Internet Protocol). In another embodiment, the network 526 may be a LAN or a WAN. In another embodiment, the network 526 may be a hotspot service provider network. In another embodiment, the network 526 may be an intranet. In another embodiment, the network 526 may be a GPRS (General Packet Radio Service) network. In another embodiment, the network 526 may be any appropriate cellular data network or cell-based radio network technology. In another embodiment, the network 526 may be an IEEE 802.11 wireless network. In still another embodiment, the network 526 may be any suitable network or combination of networks. Although one network 526 is shown, in other embodiments any number of networks (of the same or different types) may be present.

It should be understood that the various techniques described herein may be implemented in connection with hardware or software or, where appropriate, with a combination of both. Thus, the methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computer, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or use the processes described in connection with the presently disclosed subject matter, e.g., through the use of an API, reusable controls, or the like. Such programs may be implemented in a high level procedural or object-oriented programming language to communicate with a computer system.

However, the program(s) can be implemented in assembly or machine language, if desired. In any case, the language may be a compiled or interpreted language and it may be combined with hardware implementations. Although exemplary embodiments may refer to using aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be spread across a plurality of devices. Such devices might include personal computers, network servers, and handheld devices, for example.

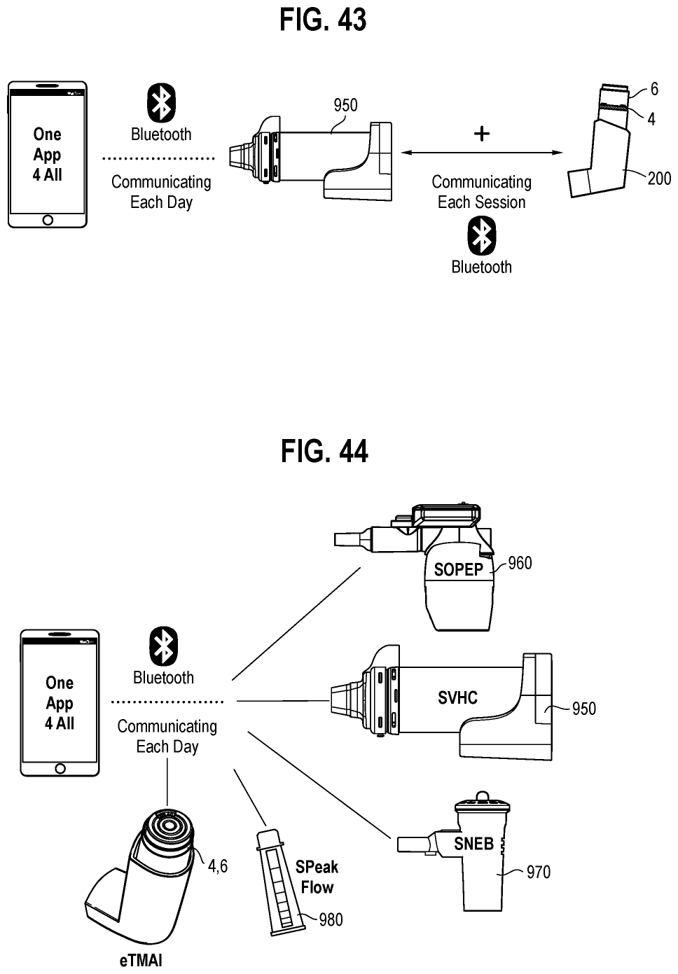

Referring to FIGS. 43-45, the eTMAI 4, 6 is designed such that it may communicate with other nearby devices if required, and may include one or more connection protocols, including: (1) Proximity (0-10m): NFC, RFID; (2) Wireless Personal Area Network (10-100m): BLE, ZigBee, ISA100; (3) Wireless Local Area Network (100-1000m): 802.11 IEEE; (4) Wireless Neighborhood Area Network (˜5-10 km): Wi-SUN; and/or (5) Wireless Wide Area Network (up to 100 km): Cellular (LTE CAT M1, 4G, 5G, LPWAN, SigFox, LoRa).

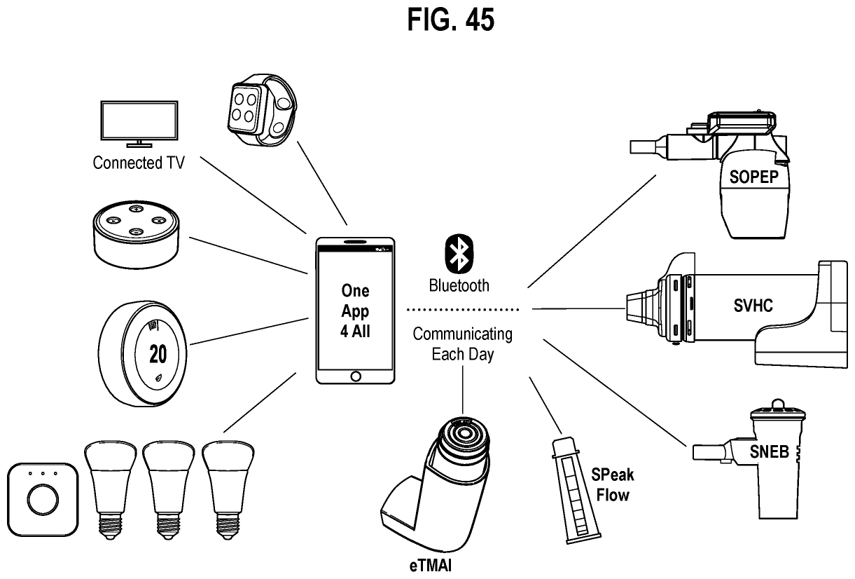

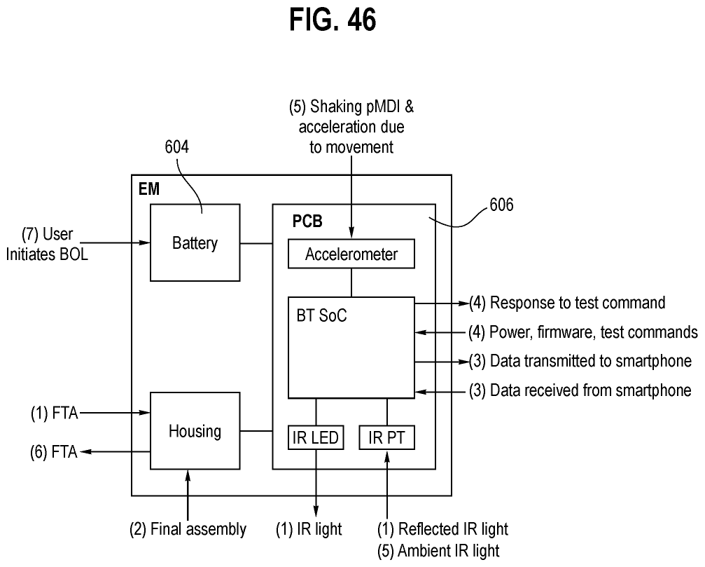

In one embodiment, shown in FIG. 43, a smart valved holding chamber 50 (SVHC) may communicate with the eTMAI 4, 6 To best confirm that the drug released by the eTMAI was inhaled, the SVHC 950 may could sense inhalation detection and inhalation completion if used in line with the eTMAI, this adherence method ensures that the medication was inhaled/delivered, and the patient took a breath in. The adherence data captured by the eTMAI and the SVHC are communicated to the SVHC Smart Phone Application where they are analyzed and displayed on the screen. Inhalation confirmation is the combination of inhalation detection and inhalation completion. The VHC/SVHC helps properly deliver the correct amount of drug to the lungs and not to the back of the throat. The sVHC may also recognize Actuation Detection, Inhalation Detection, Inhalation Completion, and provide Event Time Stamp. The combination thereby provides greater value (assurance and credibility) to the user and the adherence tracking record. At the same time, the eTMAI can recognize the drug used and the number of doses left in the canister. Referring to FIG. 44, the eTMAI is connected to the Smartphone Application and the other related medical devices, which may include a smart oscillating positive expiratory pressure device 960, a smart nebulizer device 970, a smart valved holding chamber 950, and/or a smart peak flow device 980. The eTMAI is able to connect to a range of other smart devices in the close vicinity as such. In an exemplary connected environment, a virtual assistant may be connected to provide reminders to the user to take a certain medication, or a Philips Hue programed light may provide a visual output, e.g., a certain color at a certain time, as a reminder of what MDI drug to take. Alternatively, a smart watch may provide reminders regarding the time and type of medication to take.

### Shake Detection:

For some drug formulations, it may be desirable to shake the MDI before actuation to ensure the correct dose is emitted during each actuation. As such, in one embodiment, a system includes an algorithm to detect if the inhaler was shaken prior to actuation. For some medications, the delay between shake and actuation may have an impact on the next dose, so the time between a shake and an actuation may also be considered in the shake detection algorithm.

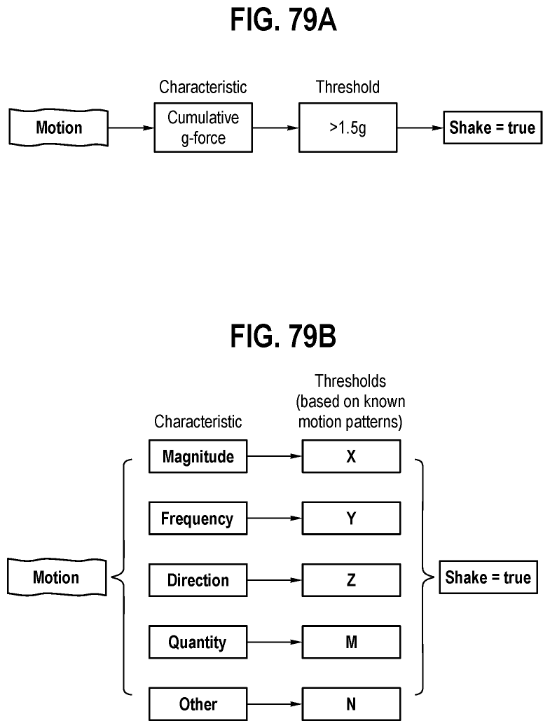

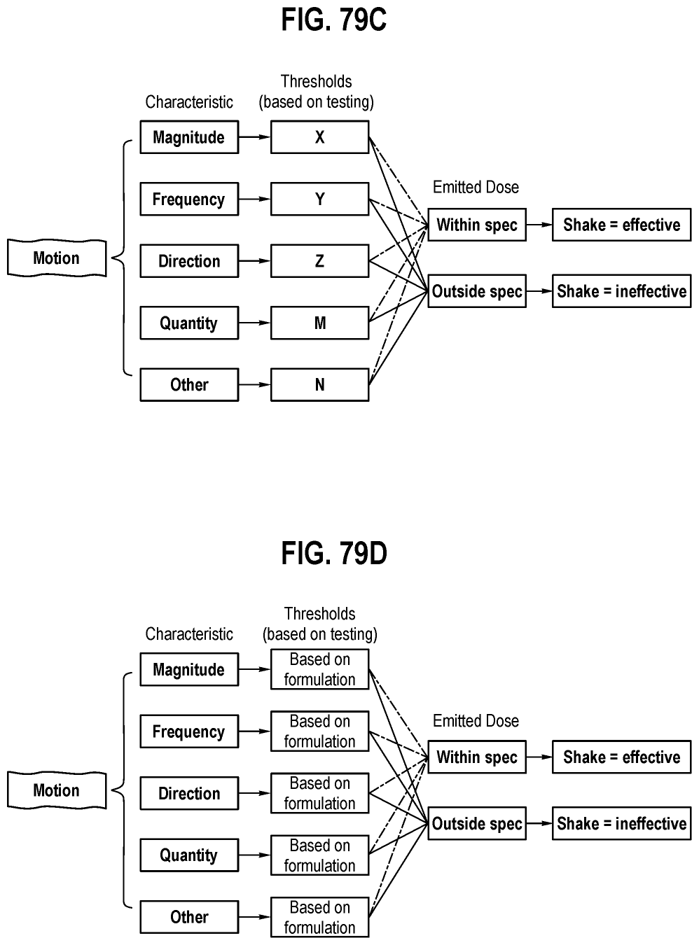

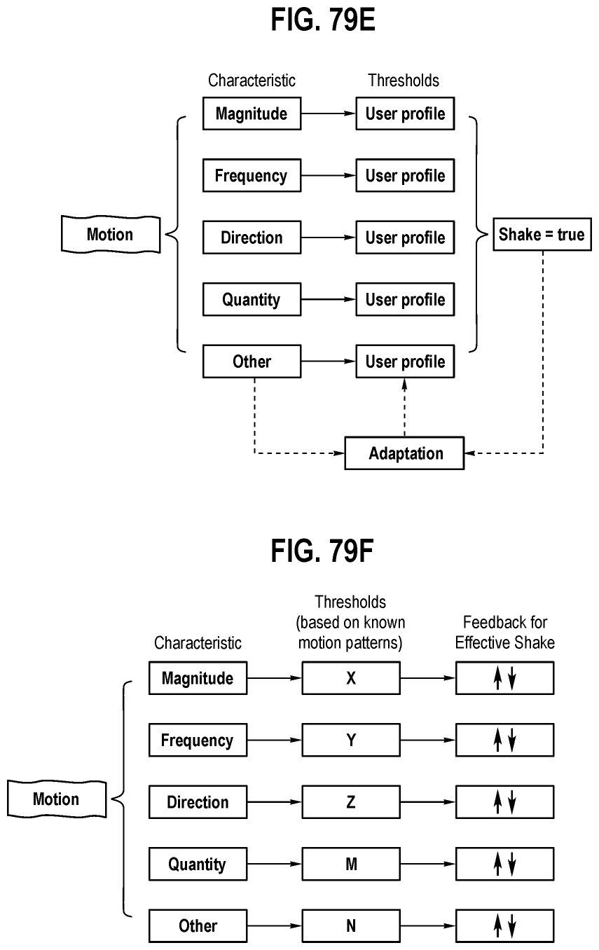

In one embodiment, as shown in FIG. 79A, a simple shake detection algorithm includes measuring the cumulative g-force from an accelerometer, determining whether the measure force crossed a predetermined threshold (e.g., 1.5 g), and then flagged or provided notice that the MDI had been suitably shaken. If the MDI was then actuated within a predetermined time period (e.g., 10 sec) of being flagged as shaken, then that actuation was also flagged as being shaken.

In another embodiment, shown in FIG. 79B, motion is measured using an accelerometer and a shake detection algorithm takes into account other variables, besides cumulative g-force, such as magnitude, frequency, direction, and quantity of shake maneuvers, which may be evaluated and used to develop a shake pattern. The recorded shake pattern is compared to known patterns and classified as a shake or not. Machine learning is well suited to this type of classification algorithm, provided sufficient ‘known’ data is available to train the algorithm. This type of algorithm only looks to identify if the inhaler was shaken and does not consider the effectiveness of the shake.

In another embodiment, shown in FIG. 79C, the effectiveness of the shake is considered. To develop the algorithm, shake characteristics such as magnitude, frequency, direction, and quantity are controlled and varied and their impact on emitted dose is quantified. An algorithm is then developed which evaluates the motion data (magnitude, freq, direction, quantity) and determines if it is sufficient to ensure an effective emitted dose.

In another embodiment, shown in FIG. 79D, the above algorithm is adapted for each drug and/or formulation. Different formulations and/or drugs may require different levels of shaking to ensure proper emitted doses. Using the drug information stored in the app (via the QR code), the algorithm adapts to the thresholds for the specific formulation being used.

In another embodiment, shown in FIG. 79E, a customized shake profile is created for each patient during initial setup. The algorithm then sets the thresholds based on the user's profile. Overtime, the algorithm continually adapts the user's shake thresholds based on new inputs and confirmed shake events.

In another embodiment, shown in FIG. 79F, a training mode is included in the app where feedback is provided to the user to learn how to shake their inhaler. The user shakes the inhaler and in real-time the app provides feedback on what changes may be needed to produce an effective shake.

### Alternative Feedback Embodiments

In another embodiment, a haptic feedback module may be located inside the eTMAI, producing a vibration notifying the user by buzzing at different frequencies, which may be programmed by the user in an application settings.

In other embodiments, speakers may provide auditory or sound feedback, including for example musical tones, and/or a speaking voice notifying user with activity events such as: (a) One buzzing or beep, good technique, accompanied by a green LED, or vice versa; (b) Two buzzing or beeps, poor technique, accompanied by a red LED, or vice versa; (c) Buzzing with a tone or melody (programmable via app), reminding user to take their medication at pre-set times.

In other embodiments, a scent emitting system may provide olfactory or smell feedback, including a device to emit a scent A if drug A is used and a scent B if drug B is used. The scent emitter may be a scratch sniff label installed by the manufacture of the medicament. The scent emitting system may be especially beneficial to hearing or vision impaired individuals when identifying the medicament being dispensed by the MDI.

In another embodiment, the device may be configured with a brail indicator 804, for example including On/OFF protruding features, or indicate in brail a character representing Drug A, and another character representing Drug B canisters by touch.

In another embodiment, the system may be configured with a locater device, activated for example by pressing an icon on an application to locate the nearby inhaler, or by incorporating hardware in the system that is reactive to auditory inputs, such as whistling or clapping, with the device emitting, e.g. with a microphone, an auditory output or signal, for example a whistle or return sound.

### Alternative Systems Operations:

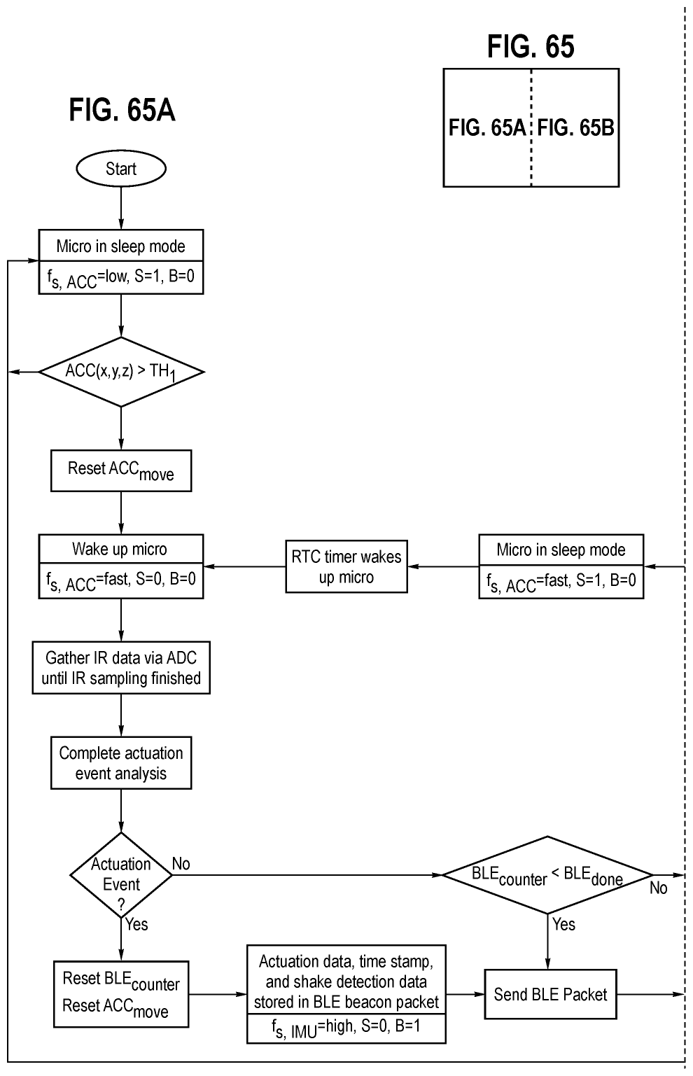

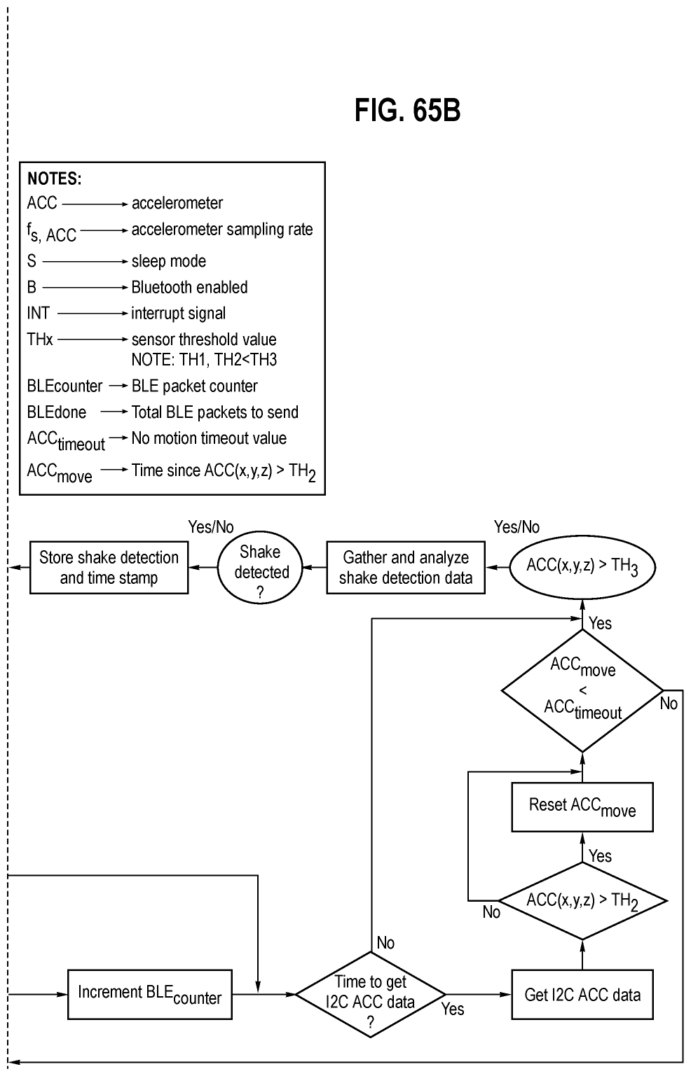

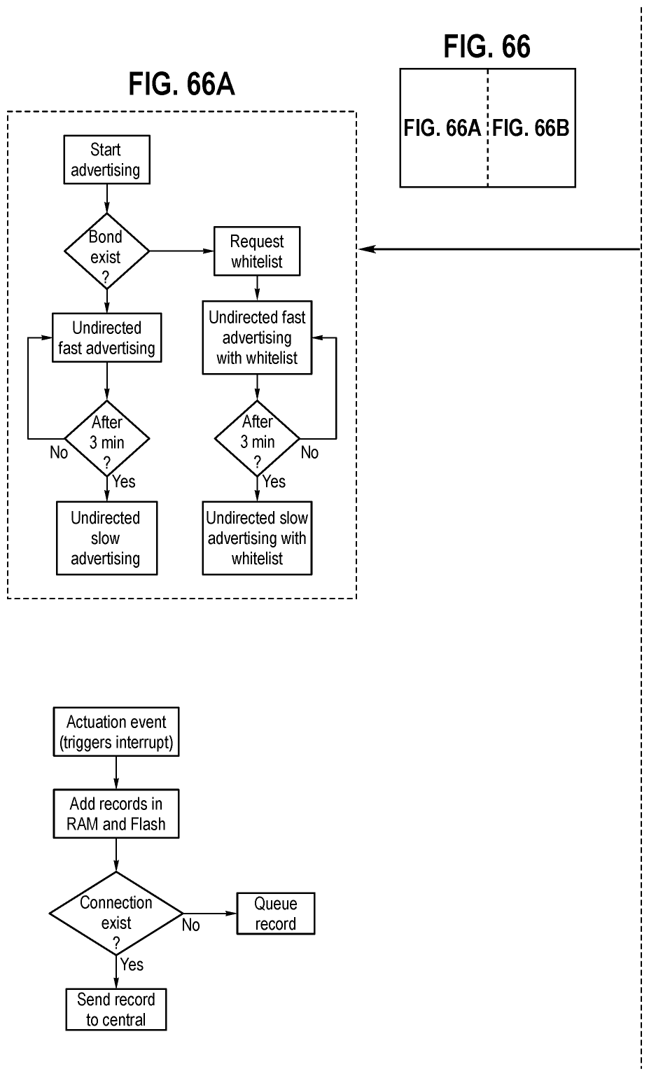

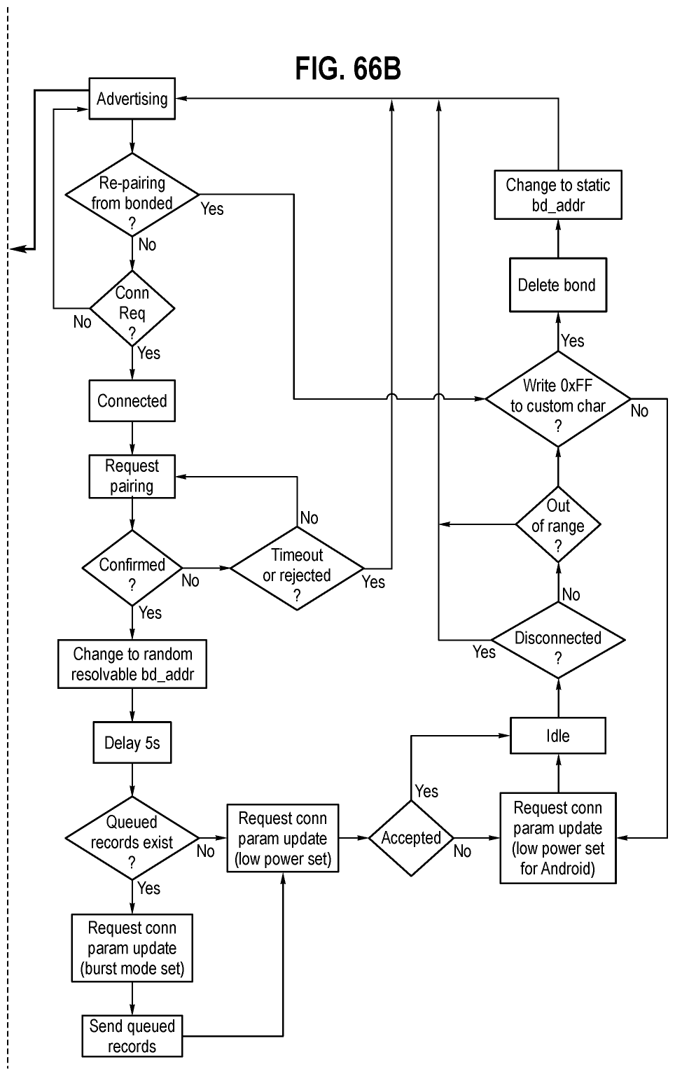

Referring to FIG. 65, operation of the system progresses through an alternative order of events, including a) check for actuation event, b) send Bluetooth packet, c) check timer to get accelerometer data, d) check for shake, and e) sleep. This order reduces the delay in getting the actuation detection sample. Due to limitations relative to the coin cell battery, it may not be feasible to have both BLE transmission and IR actuation detection occurring at the same time. As such, the firmware logic prioritizes actuation detection and prevents BLE transmissions while checking for actuation events.

### Bluetooth Low Energy Logic:

In one embodiment, the BLE connection operates in ‘Beacon’ mode, wherein the eTMAI is not connectable and only sends undirected advertisements so that any phone can read the data packets. Alternatively, the eTMAI acts in a BLE peripheral role and the smartphone acts as the central role. The eTMAI may start in advertising mode while waiting for a connection request from the central device. Once connected, the eTMAI initiates pairing and bonding. Pairing is the process where devices exchange the necessary information to establish a connection and bonding stores the pairing information on the device so that the pairing process does not have to be repeated every time the devices reconnect to each other.

When there is no active BLE connection with a central device, the eTMAI is put into advertising mode to make itself discoverable by a nearby central device. Depending on whether there is a previously bonded central device, this advertising can be directed or undirected. If there is no previously bonded central device, the eTMAI goes into general discoverable mode and sends undirected, connectable advertising packets so that it is discoverable and connectable by all central devices within range. If the eTMAI was previously bonded with a central device, it will try to reconnect to this known device, by going to directed connectable advertising mode. During this time, the eTMAI is not scannable by any central devices other than the known peer device. If the central device is in range, the central device will find and auto reconnect to this eTMAI device. Previous bonding information will be reused, and no pairing procedure is needed to form a secure connection.

Should the user need to connect to the eTMAI with another central device, the bonding information may need to be deleted. When the bond is deleted or reset, the eTMAI goes into general discovery and undirected connectable modes. In one embodiment, the user may delete the bond in the app, but in case of a lost or stolen phone, a reset procedure may be required on the device.

### QR Code:

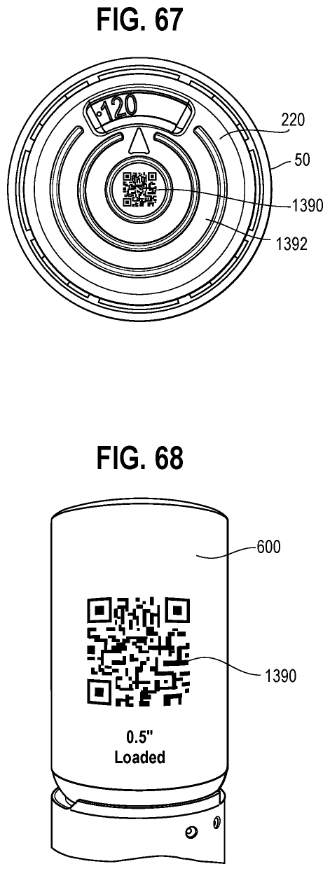

Referring to FIGS. 67 and 68, various embodiments of the eTMAI may use a QR code 1390 during the pairing process to achieve better wireless security and a better user experience. In one embodiment, here are two types of information that may be embedded into the QR code: BLE information and Drug information.

### BLE Information:

Information that is exchanged between the peripheral and the central devices during the pairing process may be intercepted and used to compromise security. This is known as a man-in-the-middle attack. To address this issue, one embodiment of the BLE protocol allows for other means of communication to exchange sensitive information used to establish a secure connection. This is called out-of-band (00B) pairing and it uses technology such as NFC or QR codes 1390. In one embodiment, a QR code 1390 may be used to embed sensitive information, which information may be scanned by the central device (e.g., phone) during the pairing process. The phone reads the QR code and uses the information to complete the pairing process.

### Drug Information:

Other information may also be embedded into the QR code 1390. In one embodiment, drug information including but not limited to: drug name, drug class, dose strength, expiry, manufacturer, and lot number may be embedded into the QR and that information may be read by the phone and auto populated in the app, thereby greatly increasing the user experience. The QR code may be applied to one or both of the mechanical dose counter and the retaining member. In one embodiment, shown in FIG. 67, the QR code is applied to a top surface 1392 of the mechanical dose counter. In another embodiment, shown in FIG. 68, the QR code is applied to a label wrap 600 surrounding at least peripheral portions of the mechanical dose counter and/or retaining member. The QR code 1390 may be printed or lasered on the eTMAI, or the components thereof. For example, in the embodiment of FIG. 67, the QR code is lasered onto the top surface 1392.

### QR Code—Manufacturing Process:

In some embodiments, for example when the QR code 1390 is printed, one challenge relates to transferring the data required for the QR code between the device manufacturer and the drug manufacturer in a way that is suitable for high-volume manufacturing but provides a unique signature for each device (e.g., BLE info is unique for each device while the drug info may not be). The process involves interaction between the device manufacture, the drug manufacturer and the end user (e.g., patient).

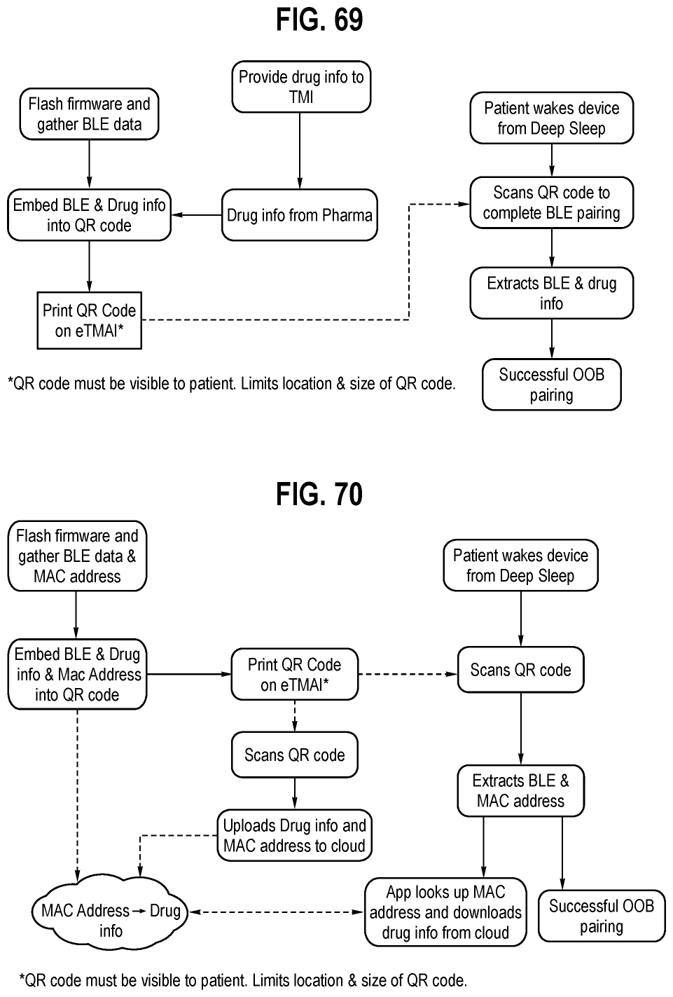

In one embodiment, shown in FIG. 69, the device manufacturer generates and prints the QR code 1390 directly onto the eTMAI device (e.g., cap or base) using drug information provided by the drug manufacturer and BLE information extracted from the firmware. In this embodiment, the drug information needs to be provided before the eTMAI devices are manufactured so that the information may be embedded into the QR code 1390. One benefit of this embodiment is that there are no new assembly steps for the drug manufacturer. Since the QR code 1390 is being applied (e.g., printed or lasered) directly on the eTMAI, this option may limit the location and size of the QR code because the QR code must be visible after the label is applied. A smaller QR code may limit the amount of information that can be embedded.

In another embodiment, shown in FIG. 70, the device manufacturer gathers the BLE information and a MAC address and embeds it into a QR code 1390 which is printed onto the eTMAI. The MAC address is a unique number for each unit. The MAC address also is uploaded into a cloud database. During assembly of the MDI, the drug manufacturer may scan the QR code and upload the matching drug information to the cloud. The cloud database contains the MAC address for each unit and the corresponding drug information. During initial setup, the user may scan the QR code that contains the BLE information needed to achieve a successful OOB pairing. The app will then use the MAC address, which is also embedded in the QR code, to perform a lookup operation and download the matching drug information. In this embodiment, the drug information does not need to be assigned to the eTMAIs until they are attached to the MDI, which may reduce the planning effort and inventory management. In this embodiment, the drug manufacturer will need to scan the QR code and upload the drug info. Moreover, in this embodiment, the location and size of the QR code may be limited, thereby also limiting the amount of data in the QR code.

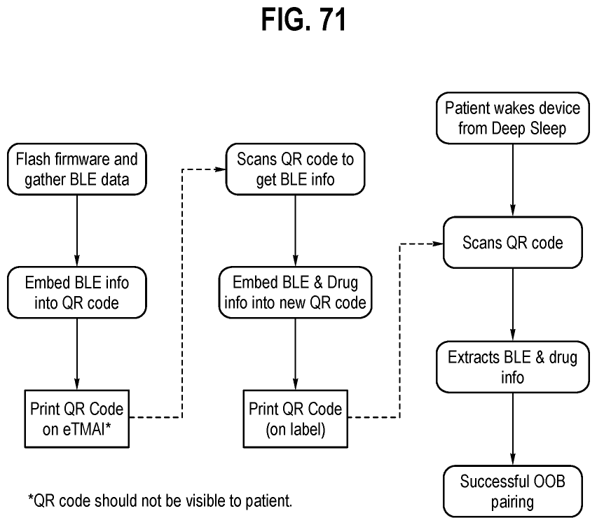

In another embodiment, shown in FIG. 71, two QR Codes may be used: one to communicate between the device manufacturer and the drug manufacturer, and one to communicate with the end-user. The first QR code is generated by the device manufacturer and contains the BLE information. The QR code is preferably applied to a location that the end user cannot see, for example the bottom of the eTMAI, which is later coupled to the medicament container thereby covering the first QR code. During assembly to the MDI, the drug manufacturer scans the QR code on the device to extract the BLE information, then generates a new QR code that includes both the BLE information and the drug information. Next, the second QR code 1390 is printed onto the label 600, which is then wrapped around the eTMAI and MDI canister during assembly. During initial setup, the end user scans the QR code 1390 on the label and extracts the drug information and achieves a successful OOB paring. In this embodiment, the QR code may be larger, for example providing a 0.50 to 0.75 inch field, which allows more information to be embedded and may be easier to scan. One challenge with this embodiment is that each unit must be tracked during the assembly process to ensure the correct label (with QR code) is used with the correct eTMAI.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. An indicating device comprising:
a mechanical dose counter adapted to count the number of doses that have been dispensed from or remain in a container;
a printed circuit board assembly adapted to record when the doses have been dispensed from the container;
a battery electrically connectable to the printed circuit board assembly; and
a retaining member coupled to the mechanical dose counter, wherein the retaining member sandwiches the printed circuit board assembly between the retaining member and the mechanical dose counter.

2. The indicating device of claim 1 wherein the retaining member comprises a ring portion surrounding the battery and printed circuit board assembly and a plurality of arms extending upwardly from the ring portion and engaging the mechanical dose counter.

3. The indicating device of claim 2 wherein the printed circuit board assembly comprises a printed circuit board with a plurality of cutouts mating with arms.

4. The indicating device of claim 2 wherein at least one of the arms comprises a catch portion engaging the mechanical dose counter.

5. The indicating device of claim 4 wherein the catch portion engages an edge of a bottom wall of the mechanical dose counter, and wherein the bottom wall comprises at least one opening mating with and receiving at the at least one of the arms.

6. The indicating device of claim 1 further comprising a QR code applied to one or both of the mechanical dose counter and retaining member.

7. The indicating device of claim 6 wherein the QR code is applied to a top surface of the mechanical dose counter.

8. The indicating device of claim 6 wherein the QR code is applied to a wrap surrounding at least peripheral portions of the mechanical dose counter and/or retaining member.

9. An indicating device comprising:
a mechanical dose counter adapted to count the number of doses that have been dispensed from or remain in a container, wherein the mechanical dose counter comprises a base and a cap moveable relative to the base between a first position and a second position;
a printed circuit board assembly adapted to record when the doses have been dispensed from the container;
a battery electrically connectable to the printed circuit board assembly; and
a contact mechanism disposed between the battery and the printed circuit board, wherein the contact mechanism is moveable between a first position wherein the battery is not electrically coupled to the printed circuit board assembly and a second position wherein the battery is electrically coupled to the printed circuit board.

10. The indicating device of claim 9 wherein the contact mechanism comprises a switch, wherein the switch is moveable between the first and second positions in response to the movement of the cap relative to the base between the first and second positions.

11. The indicating device of claim 10 wherein the switch comprises a pair of spring fingers, wherein the cap and/or the base biases one of the spring fingers into contact with the other of the spring fingers as the cap is moved relative to the base between the first and second positions.

12. The indicating device of claim 11 wherein the spring fingers remain in contact after a first movement of the cap relative to the base between the first and second positions.

13. The indicating device of claim 10 wherein the contact mechanism comprises a slide member, wherein the cap and/or the base moves the slide member to close a circuit between the battery and the printed circuit board as the cap moves relative to the base between the first and second positions.

14. The indicating device of claim 10 wherein the switch comprises a reed switch, and wherein at least one of the cap or base comprises a magnet, wherein the magnet closes the reed switch as the cap moves relative to the base between the first and second positions.

15. The indicating device of claim 9 wherein the contact mechanism comprises a pull tab.

16. The indicating device of claim 15 wherein the pull tab comprises a first portion disposed between a pair of contacts, and a second portion disposed exteriorly to the mechanical dose counter.

17. The indicating device of claim 16 wherein the second portion is adhered to the cap.

18. The indicating device of claim 16 wherein the pair of contacts comprises the battery and a contact on the printed circuit board.

19. The indicating device of claim 16 wherein the pair of contacts comprises a pair of spring fingers.

20. The indicating device of claim 9 wherein the contact mechanism comprises a snap member moveable between the first and second positions.

21. The indicating device of claim 20 wherein the snap member comprises a beam moveable overcenter between the first and second positions.
